(19) United States
(12) Patent Application Publication
(10) Pub. No.: US 2025/0366764 A1
(43) Pub. Date: Dec. 4, 2025

(54) PHYSICAL CONDITION DETECTION DEVICE, PHYSICAL CONDITION DETECTION SYSTEM, PHYSICAL CONDITION DETECTION METHOD, AND STORAGE MEDIUM STORING PHYSICAL CONDITION DETECTION PROGRAM

(71) Applicant: Mitsubishi Electric Corporation, Tokyo (JP)

(72) Inventors: Takeru SHIRAGA, Tokyo (JP); Shunya OSAWA, Tokyo (JP)

(73) Assignee: Mitsubishi Electric Corporation, Tokyo (JP)

(21) Appl. No.: 19/306,481

(22) Filed: Aug. 21, 2025

Related U.S. Application Data
(63) Continuation of application No. PCT/JP2023/007089, filed on Feb. 27, 2023.

(51) Int. Cl. A61B 5/352; A61B 5/024

(52) U.S. Cl. CPC A61B 5/352; A61B 5/024

(57) ABSTRACT

A physical condition detection device determines a change in physical condition of a subject as an inspection target user, includes processing circuitry to construct a reference of a pulsation interval of each of users as a reference pulsation model in regard to each user, to select the reference pulsation model of the subject from the reference pulsation models and to generate a reference value indicating the pulsation interval of the subject from the selected reference pulsation model, to receive time-series pulsation information being information regarding the subject detected by a sensor, to extract a feature value of a real-time pulsation interval from the time-series pulsation information, and to correct the feature value by normalizing the feature value by using the reference value, thereby generating a real-time corrected feature value, and to determine a change in the physical condition of the subject based on the real-time corrected feature value.

### CROSS-REFERENCE TO RELATED APPLICATION

This application is a continuation application of International Application No. PCT/JP2023/007089 having an international filing date of Feb. 27, 2023.

### BACKGROUND OF THE INVENTION

### 1. Field of the Invention

The present disclosure relates to a physical condition detection device, a physical condition detection system, a physical condition detection method and a physical condition detection program.

### 2. Description of the Related Art

There has been proposed a device that detects a change in physical condition such as an irregular pulse by using information regarding the interval of pulsation of the heart (i.e., pulsation interval) that can be measured as the heart rate or the pulse. The device using the pulsation interval alone as the input has advantages in that the amount of data to be processed is small and the measurement load is low compared to devices using electrocardiogram information obtained by an electrocardiograph. Further, the device using the pulsation interval alone as the input has an advantage in that a change in physical condition can be detected by using the pulsation interval sensed by a noncontact sensor not in contact with a user as a subject or the pulsation interval measured by a wearable device worn by the user.

For example, Patent Reference 1 discloses a technology in which a resting state heart rate is identified by calculating a representative value of the heart rate from pulsation information measured when the user is in the resting state and the condition of the user is calculated by using a feature value calculated by using the resting state heart rate and a heart rate observed in real time.

Further, Patent Reference 2 discloses a technology of detecting atrial fibrillation by using an electrocardiogram measured at a time separate from the system operation and cardiac activity information including the pulsation interval acquired from a wearable PPG (PhotoPlethysmoGraphy) sensor.

Patent Reference 1: Japanese Patent Application Publication No. 2020-92804.

Patent Reference 2: Japanese Patent Application Publication No. 2020-506770.

The technology disclosed in the Patent Reference 1 uses the median of the resting state heart rate as information for correcting individual difference occurring in the pulsation interval. However, variance occurs in the heart rate even in cases of assuming the same user, and thus in the case of handling the median of the resting state heart rate, being a single item of scalar information, as a reference value, there is a possibility that the pulsation interval as the reference of each user cannot be grasped accurately. Therefore, the technology disclosed in the Patent Reference 1 has insufficient accuracy as a method for detecting a change in the physical condition (e.g., a sudden change in the physical condition, namely, a sudden deterioration in the physical condition).

The technology disclosed in the Patent Reference 2 realizes the detection of atrial fibrillation corresponding to the individual difference in the cardiac activity by correcting the cardiac activity information acquired in real time by using the previously measured electrocardiograms collected in regard to each individual and the cardiac activity information including the pulsation interval. However, the technology disclosed in the Patent Reference 2 was designed with the precondition of using an electrocardiogram measured by an electrocardiograph, and is incapable of detecting a change in the physical condition (e.g., a sudden change in the physical condition, namely, a sudden deterioration in the physical condition) based on the pulsation interval obtained by a wearable sensor or the like.

### SUMMARY OF THE INVENTION

It is therefore an object of the present disclosure to provide a physical condition detection device, a physical condition detection system, a physical condition detection method and a physical condition detection program that make it possible to accurately detect a change in the physical condition of the user by using the pulsation interval.

A physical condition detection device in the present disclosure is a device that determines a change in physical condition of a subject as an inspection target user identified by user identification information. The physical condition detection device includes processing circuitry to construct any of a reference of a pulsation interval, a reference of a feature value obtained from the pulsation interval, and both of the references of the pulsation interval and the feature value, as reference pulsation models in regard to each user; to select a reference pulsation model of the subject from the reference pulsation models and to generate any of a reference value indicating the pulsation interval of the subject, a reference value of the feature value obtained from the pulsation interval of the subject, and both of the reference values of the pulsation interval of the subject and the feature value of the subject, as a subject reference value, from the selected reference pulsation model; to receive time-series pulsation information that is information regarding the subject and is detected by a sensor, to extract a real-time feature value that is a feature value of a real-time pulsation interval from the time-series pulsation information, and to correct the real-time feature value by normalizing the real-time feature value by using the subject reference value, thereby generating a real-time corrected feature value; to determine a change in the physical condition of the subject based on the real-time corrected feature value; to determine whether update of the reference pulsation models should be made or not based on a physical condition determination result that is a result of the determining the update of the reference pulsation models; and to update the reference pulsation models in real time when it is determined that the update should be made.

A physical condition detection method in the present disclosure is a method to be executed by a physical condition detection device that determines a change in physical condition of a subject as an inspection target user identified by user identification information. The method includes constructing any of a reference of a pulsation interval, a reference of a feature value obtained from the pulsation interval, and both of the references of the pulsation interval and the feature value, as reference pulsation models in regard to each user; selecting a reference pulsation model of the subject from the reference pulsation models and generating any of a reference value indicating the pulsation interval of the subject, a reference value of the feature value obtained from the pulsation interval of the subject, and both of the reference values of the pulsation interval of the subject and the feature value of the subject, as a subject reference value, from the selected reference pulsation model; receiving time-series pulsation information that is information regarding the subject and is detected by a sensor, extracting a real-time feature value that is a feature value of a real-time pulsation interval from the time-series pulsation information, and correcting the real-time feature value by normalizing the real-time feature value by using the subject reference value, thereby generating a real-time corrected feature value; determining a change in the physical condition of the subject based on the real-time corrected feature value; determining whether update of the reference pulsation model should be made or not based on a physical condition determination result that is a result of the determining of the update of the reference pulsation model; and updating the reference pulsation model in real time when it is determined that the update should be made.

According to the present disclosure, a change in the physical condition of the user can be detected accurately by using the pulsation interval.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a diagram showing the hardware (HW) configuration of a physical condition detection system according to a first embodiment;

FIG. 2 is a block diagram showing the functional configuration of a physical condition detection device according to the first embodiment;

FIG. 3 is a diagram showing an example of sensor information inputted to the physical condition detection device according to the first embodiment;

FIG. 4 is a block diagram showing the functional configuration of a reference pulsation model construction unit of a reference pulsation model management unit in FIG. 2;

FIG. 5 is a diagram showing an example of RR intervals as information calculated from an electrocardiogram;

FIG. 6 is a diagram showing a general outline of a process flow executed by the reference pulsation model construction unit of the reference pulsation model management unit in FIG. 2;

FIG. 7 is a diagram showing details of the process flow executed by the reference pulsation model construction unit of the reference pulsation model management unit in FIG. 2;

FIG. 8 is a block diagram showing the functional configuration of a process continuation determination unit of a physical condition detection unit in FIG. 2;

FIG. 9 is a block diagram showing the functional configuration of a normalized feature extraction unit of the physical condition detection unit in FIG. 2;

FIG. 10 is a diagram showing a general outline of a process flow executed by the physical condition detection unit in FIG. 2;

FIG. 11 is a diagram showing details of the process flow executed by the physical condition detection unit in FIG. 2;

FIG. 12 is a block diagram showing the functional configuration of a physical condition detection device according to a second embodiment;

FIG. 13 is a diagram showing details of a process flow executed by the reference pulsation model construction unit of a reference pulsation model management unit in FIG. 12;

FIG. 14 is a block diagram showing the functional configuration of a process continuation determination unit and a situation determination unit of a physical condition detection unit in FIG. 12;

FIG. 15 is a diagram showing a general outline of a process flow executed by the physical condition detection unit in FIG. 12;

FIG. 16 is a diagram showing details of the process flow executed by the physical condition detection unit in FIG. 12;

FIG. 17 is a block diagram showing the functional configuration of a physical condition detection device according to a third embodiment;

FIG. 18 is a diagram showing a general outline of a process flow executed by a physical condition detection unit in FIG. 17;

FIG. 19 is a diagram showing details of the process flow executed by the physical condition detection unit in FIG. 17;

FIG. 20 is a block diagram showing the functional configuration of a physical condition detection device according to a fourth embodiment;

FIG. 21 is a diagram showing a general outline of a process flow executed by a physical condition detection unit in FIG. 20; and

FIG. 22 is a diagram showing details of the process flow executed by the physical condition detection unit in FIG. 20.

### DETAILED DESCRIPTION OF THE INVENTION

A physical condition detection device, a physical condition detection system, a physical condition detection method and a physical condition detection program according to each embodiment will be described below with reference to the drawings. The following first to fourth embodiments are just examples and it is possible to appropriately combine embodiments and appropriately modify each embodiment.

### (1) First Embodiment

In a first embodiment, a description will be given of a physical condition detection device, a physical condition detection system, a physical condition detection method and a physical condition detection program for detecting a change in the physical condition (e.g., a sudden change in the physical condition, namely, a sudden deterioration in the physical condition) of a user as a subject by using a corrected feature value obtained by correcting the influence of the individual difference in the pulsation interval on a feature value.

### (1-1) Physical Condition Detection System

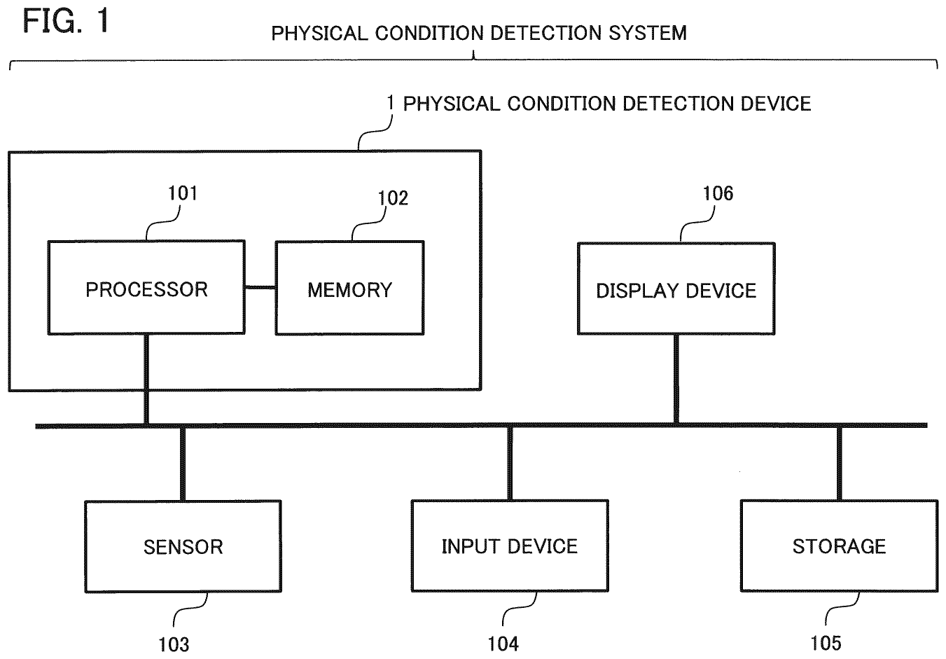

FIG. 1 is a diagram showing the HW configuration of a physical condition detection system according to the first embodiment. The physical condition detection system according to the first embodiment includes a physical condition detection device 1 and a sensor 103. The physical condition detection system according to the first embodiment may include an input device 104, storage 105 and a display device 106. The HW configuration in FIG. 1 is just an example; the HW configuration of the physical condition detection system according to the first embodiment is not limited to the example in FIG. 1.

The physical condition detection device 1 is a device capable of executing a physical condition detection method according to the first embodiment. The physical condition detection device 1 is formed with a computer, for example. The computer is a computer (e.g., a personal computer, a tablet terminal, a smartphone, a server computer on a network, or the like) capable of communicating with the sensor 103. The physical condition detection device 1 includes a memory 102 as a storage device and a processor 101 such as a CPU (Central Processing Unit) that executes a program. The program includes a physical condition detection program according to the first embodiment. The program may be provided via a network, or provided in the form of being stored in a record medium (i.e., storage medium) such as an optical disc or a magnetic disk. The storage medium is a non-transitory computer-readable storage medium storing a program such as the physical condition detection program. Further, the physical condition detection device 1 may include a GPU (Graphics Processing Unit). Furthermore, the physical condition detection device 1 may be formed with processing circuitry such as a single circuit, a combined circuit or an FPGA (Field Programmable Gate Array).

The sensor 103 is a device that acquires the pulsation. The sensor 103 includes a “pulsation sensor” that measures the heart rate or the pulse and outputs pulsation information. For example, the sensor 103 includes one out of a wearable pulse sensor that can be attached to the user as the subject, a wearable electrocardiograph, heart rate sensor or pulse sensor capable of measuring the heart rate or the pulse without making contact with the user, and so forth. Further, the sensor 103 may include an “action sensor” that measures an action taken by the user and outputs user action information as information indicating the action of the user. For example, the sensor 103 may include an RGB camera, an RGB-D (RGB-Depth) camera, an infrared camera, a motion sensor, a gesture sensor or the like as the action sensor. The sensor 103 may be either a single device or a combination of a plurality of devices. Furthermore, the sensor 103 may also be a device that differs from user to user. Moreover, the sensor 103 may include an environment sensor that measures a situation the user is in and outputs environment information.

The input device 104 is a device for receiving an input from the user, such as a keyboard, a mouse or a touch panel. The input device 104 can also be a device that receives an input by audio, a device that receives an input by a gesture, or the like.

The display device 106 is an example of an information presentation device, and is, for example, a display that presents a physical condition determination result F obtained by the physical condition detection device 1 to the user. The display device 106 can be a see-through display of an HMD (Head Mounted Display), a display of a small-sized terminal such as a smartphone, a display of a car navigation system, or the like. The see-through display is a device that displays digital content in superimposition with the real visual field by using a prism or the like. The device that presents the physical condition determination result F to the user is not necessarily limited to a display device but can also be an information presentation device of a different type using audio output, vibration output, lighting or blinking of a lamp, or the like.

The storage 105 is a storage device such as an HDD (Hard Disk Drive) or an SSD (Solid State Drive). The storage 105 stores a program, sensor data obtained by measurement by the sensor 103 (e.g., the pulsation information, the user action information, etc.), a processing result obtained by the physical condition detection device 1, and so forth. The storage 105 can also be a part of the physical condition detection device 1. Alternatively, the storage 105 can be formed with a storage area of a cloud server or the like, for example.

### (1-2) Physical Condition Detection Device 1

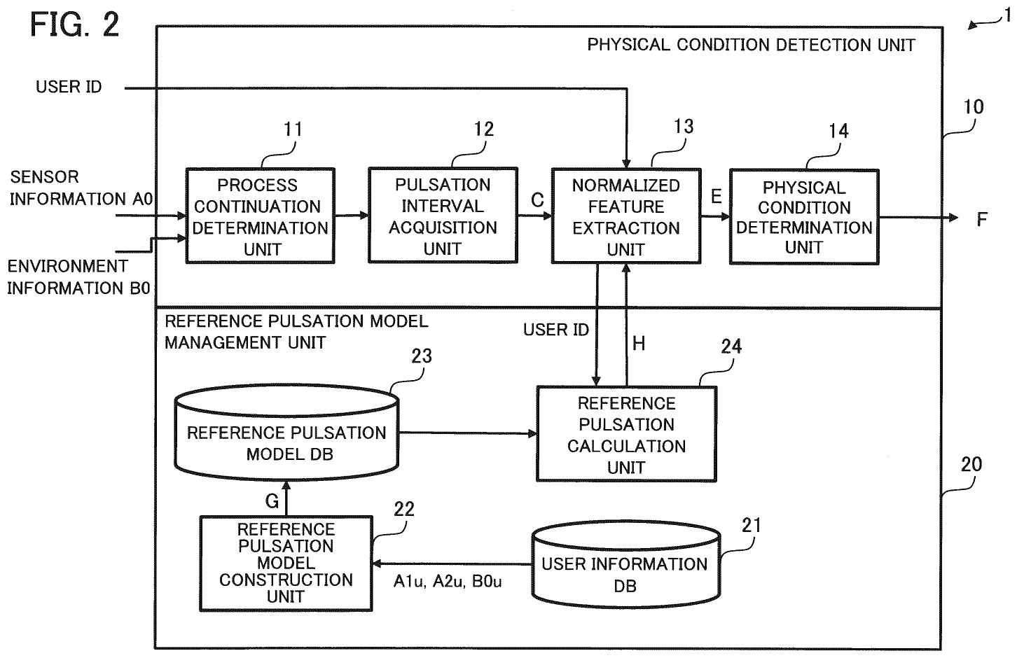

FIG. 2 is a block diagram showing the functional configuration of the physical condition detection device 1 according to the first embodiment. The physical condition detection device 1 includes a reference pulsation model management unit 20 for acquiring user information as reference information regarding each user as a subject (basic information regarding a plurality of users) and calculating a reference pulsation model G based on the user information and a physical condition detection unit 10 that receives the pulsation interval as the user's pulsation information acquired from the sensor 103, the environment information B0 indicating an environment surrounding the user, and a user ID (identifier) as user identification information as inputs and detects a change in the physical condition (e.g., a sudden change in the physical condition, namely, a sudden deterioration in the physical condition) of the user. In the first embodiment, the input of the environment information B0 is not necessarily essential. The reference pulsation model management unit 20 includes a user information database (user information DB) 21, a reference pulsation model construction unit 22, a reference pulsation model database (reference pulsation model DB) 23 and a reference pulsation calculation unit 24. The physical condition detection unit 10 includes a process continuation determination unit 11, a pulsation interval acquisition unit 12, a normalized feature extraction unit 13 and a physical condition determination unit 14. The user information DB 21 and the reference pulsation model DB 23 are stored in the storage 105 (FIG. 1), for example.

The physical condition detection device 1 is a device that determines a change in the physical condition of the subject as the inspection target user identified by the user ID, and includes the reference pulsation model construction unit 22 that constructs the reference of the pulsation interval of each of one or more users as the reference pulsation model G in regard to each user, the reference pulsation calculation unit 24 that selects the reference pulsation model of the subject from the reference pulsation models G and generates a reference value H indicating the pulsation interval of the subject from the selected reference pulsation model G, the normalized feature extraction unit 13 that receives cardiac activity information A1 as time-series pulsation information being information regarding the subject detected by the sensor 103, extracts a feature value of a real-time pulsation interval C from the time-series cardiac activity information A1, and corrects the feature value by normalizing the feature value by using the reference value H, thereby generating a real-time corrected feature value E, and the physical condition determination unit 14 that determines a change in the physical condition of the subject based on the real-time corrected feature value E.

The reference pulsation model construction unit 22 may construct the reference of the pulsation interval of each user and the situation each user is in as the reference pulsation model G in regard to each user, and the reference pulsation calculation unit 24 may select the reference pulsation model of the subject from the reference pulsation models G and generate a reference value H indicating the pulsation interval of the subject and the situation the subject is in from the selected reference pulsation model G.

Further, the reference pulsation model construction unit 22 may construct the reference of the pulsation interval of each user and the user action information indicating the action of each user as the reference pulsation model G in regard to each user, and the reference pulsation calculation unit 24 may select the reference pulsation model of the subject from the reference pulsation models G and generate the reference value H indicating the pulsation interval of the subject and the action of the subject from the selected reference pulsation model G.

The reference pulsation model construction unit 22 may construct the reference of the pulsation interval of each user, the situation each user is in, and the user action information indicating the action of each user as the reference pulsation model G in regard to each user, and the reference pulsation calculation unit 24 may select the reference pulsation model of the subject from the reference pulsation models G and generate the reference value H indicating the pulsation interval of the subject, the situation the subject is in, and the action of the subject from the selected reference pulsation model G.

### User ID

The user ID is identification information for identifying the user as the subject using the physical condition detection device 1. The user ID can be in any format as long as the user ID represents a unique value for identifying the user. The user ID is used when constructing the reference pulsation model G of each user and when calculating the reference value H from the reference pulsation model G, for example.

### Sensor Information A0

Sensor information A0 is information acquired from the sensor 103. The sensor information A0 includes the cardiac activity information A1 as the pulsation information such as a pulse wave or an electrocardiogram from which the pulsation interval can be obtained. Further, the sensor information A0 may include the user action information A2 as information in which the action of the user can be referred to (i.e., information indicating the action of the user). Examples of the user action information A2 include video data in which the user has been captured, gesture information for grasping the user's body motion, skeletal structure information regarding the user, motion capture information in which the user has been captured, and so forth.

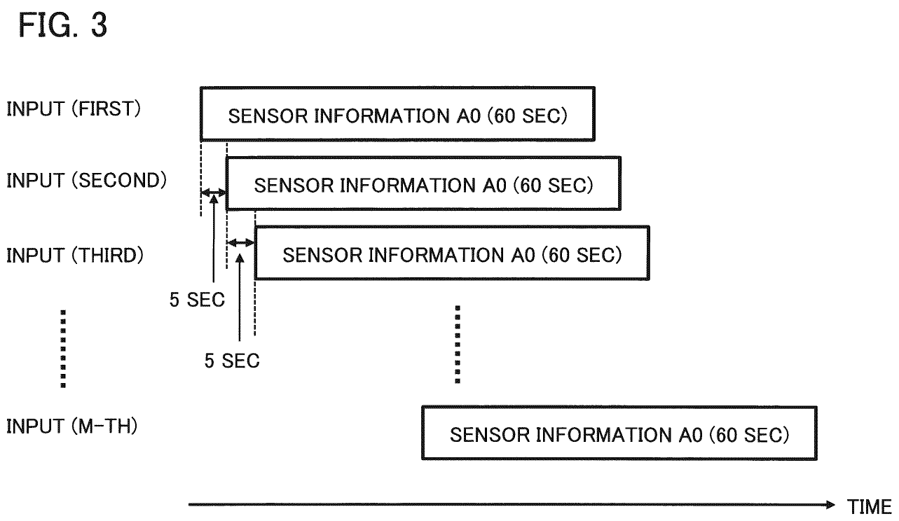

FIG. 3 is a diagram showing an example of the sensor information A0 inputted to the physical condition detection device 1 according to the first embodiment. The sensor information A0 is provided as time-series information. The data length of data inputted to the physical condition detection device 1 at a time is fixed, and the data length can be set arbitrarily. For example, when the data length is set at 60 seconds, the sensor information A0 for 60 seconds is provided as one input. Further, the frequency of the input of the sensor information A0 to the physical condition detection device 1 can be set arbitrarily. For example, when the data length is set at 60 seconds and the input frequency is set at every 5 seconds (i.e., once in 5 seconds), the sensor information A0 for 60 seconds is successively inputted to the physical condition detection device 1 every 5 seconds as indicated as FIRST, SECOND, . . . , M-TH in FIG. 3. M represents a positive integer.

Incidentally, the data length [sec] and the input frequency [times/sec] may be changed automatically or by the user's manual setting depending on the type of data included in the sensor information A0. For example, the cardiac activity information A1 as the pulsation information for calculating the pulsation interval may be set to be inputted at the data length of 60 seconds and the input frequency of “once/5 sec”, and the user action information A2 (e.g., video information) may be set to be inputted in units of frames of the video.

### Environment Information B0

The environment information B0 is information indicating the environment or situation surrounding the user as the subject. That is, the environment information B0 is information indicating the situation of the place the user is in. When the physical condition detection device 1 is used in an automobile, the environment information B0 is an in-vehicle image, GNSS (Global Navigation Satellite System) information such as GPS (Global Positioning System) information indicating the position of the automobile, CAN (Controller Area Network) data indicating driving condition of the automobile, or the like, for example. When the physical condition detection device 1 is used in a room in a building, the environment information B0 is the temperature in the room, the humidity in the room, both of the temperature and the humidity in the room, or the like, for example.

### Physical Condition Determination Result F

The physical condition determination result F obtained by the physical condition detection device 1 is a result indicating a change in the physical condition (e.g., a sudden change in the physical condition, namely, a sudden deterioration in the physical condition) of the user determined by the physical condition detection device 1. The physical condition determination result F is, for example, a value indicating the presence/absence of a sudden change in the physical condition by one of two values like “0” and “1”. The physical condition determination result F can also be a value including a decimal fraction between 0 and 1 (e.g., continuous value) as a value indicating the probability that a sudden change in the physical condition has occurred. For example, one value of the physical condition determination result F is calculated for one input of the pulsation interval C calculated from the sensor information A0. When the data length is set at 60 seconds and the input frequency is set at every 5 seconds for the sensor information A0 for calculating the pulsation interval C, the physical condition detection device 1 receives the pulsation interval data for 60 seconds and outputs the physical condition determination result F every 5 seconds.

### (1-3) Reference Pulsation Model Management Unit 20

The reference pulsation model management unit 20 is a system that generates, manages and acquires information regarding the user's pulsation interval at a time with no sudden change in the physical condition. The time with no sudden change in the physical condition means a time when no sudden change has occurred in the physical condition. The time with no sudden change in the physical condition is referred to also as a non-paroxysmal time, a normal time or a resting time. The physical condition detection unit 10 calculates a feature value, in which the individual difference has been removed from the pulsation interval, based on the reference value H calculated by the reference pulsation model management unit 20. Information regarding the pulsation interval is, for example, pulsation interval information as information indicating the pulsation interval (i.e., a value indicating the pulsation interval) such as a value itself of the pulsation interval (e.g., information such as the heat rate, a pulse rate, a heartbeat interval, a pulse interval or an RRI) or a variety of index obtained from the pulsation interval (e.g., a feature value such as a mean value or a standard deviation of the pulsation interval). The RRI is an interval from an R peak of a QRS wave to an R peak of the next QRS wave, namely, an RR interval (RR_interval). The QRS wave is a waveform that appears when a cardiac ventricle is excited.

The reference pulsation model management unit 20 constructs the reference pulsation model G for each user based on the user information DB 21 storing information such as a previously measured pulsation interval of each user and stores information regarding the reference pulsation model G in the reference pulsation model DB 23. The reference pulsation calculation unit 24 calculates the reference value H regarding the user using the physical condition detection unit 10 based on the reference pulsation model DB 23 and passes the reference value H to the physical condition detection unit 10.

### User Information DB 21

The user information DB 21 is a database storing the cardiac activity information A1 (e.g., pulsation information indicating the cardiac activity such as the pulse wave or the electrocardiogram) at the time with no sudden change in the physical condition regarding each user to use the physical condition detection device 1 (e.g., a plurality of users scheduled to use the physical condition detection device 1). The pulsation information indicating the cardiac activity stored in the user information DB 21 can include pulsation interval information obtained from the cardiac activity information A1, instead of or in addition to the cardiac activity information A1. The user information DB 21 stores the pulsation information indicating the cardiac activity (e.g., the cardiac activity information A1 or the pulsation interval information obtained from the cardiac activity information A1) as time-series information associated with the user ID.

Further, the user information DB 21 may store not only the pulsation information (e.g., the cardiac activity information and the pulsation interval information) but also the environment information B0 measured/recorded at the same time as the cardiac activity information or the pulsation interval information, or data measured/recorded at the same time as the pulsation information, from which the user action information A2 can be extracted. Examples of the data from which the user action information A2 can be extracted can include video data, motion sensor data, gesture data, biological information other than the cardiac activity information A1, and so forth. Normally, the information stored in the user information DB 21 is information already measured before the user executes the physical condition detection by use of the physical condition detection device 1. Further, the information stored in the user information DB 21 is desired to be measured in a situation similar to the situation in which the physical condition detection by the physical condition detection device 1 is executed. When the physical condition detection by the physical condition detection device 1 is supposed to be used while the user is driving an automobile, it is desirable to collect the user information during the traveling of the automobile before using the physical condition detection by the physical condition detection device 1. Furthermore, the information stored in the user information DB 21 may also be collected as data to be stored in the user information DB 21 as a part of the physical condition detection device 1 in the form of calibration of the user. Moreover, the information stored in the user information DB 21 is desired to be measured in a situation the same as the situation in which the physical condition detection by the physical condition detection device 1 is used. However, the information stored in the user information DB 21 does not necessarily have to be collected in a situation the same as the situation in which the physical condition detection by the physical condition detection device 1 is used but can be the pulsation information measured in a different situation.

Incidentally, every item of information included in the user information DB 21 is associated with a user ID and stored in a format that enables information retrieval and extraction by using a user ID.

Further, in the present application, the pulsation information relevant to the pulsation interval is referred to as the “cardiac activity information”, information indicating the measurement environment of the cardiac activity information is referred to as the “environment information”, and information relevant to the user's action when measuring the cardiac activity information is referred to as the “user action information”.

### Reference Pulsation Model Construction Unit 22

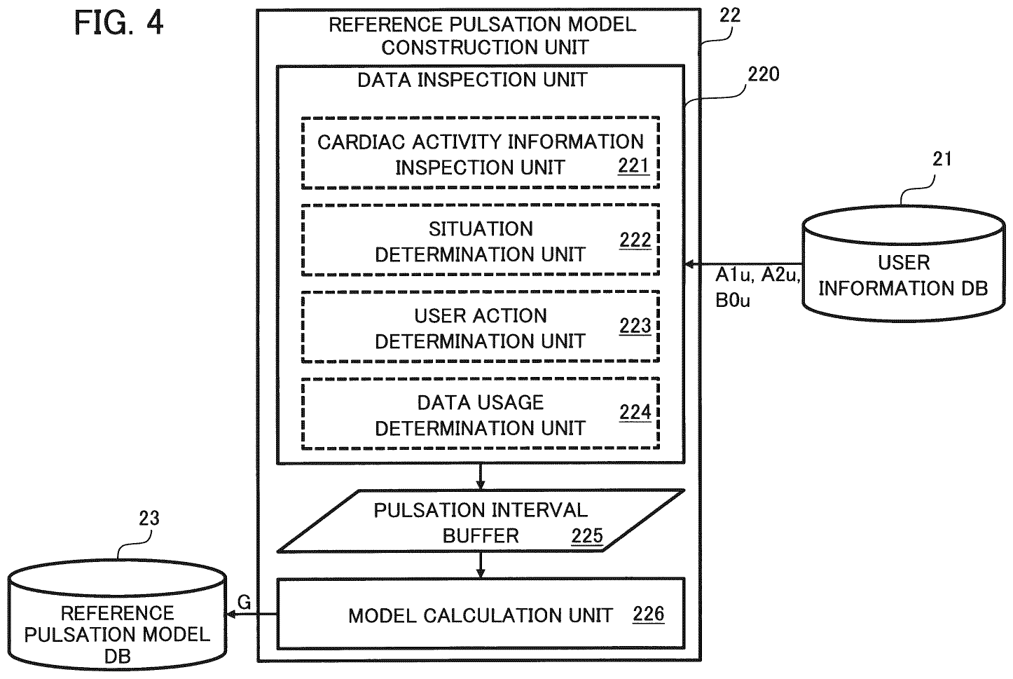

FIG. 4 is a block diagram showing the functional configuration of the reference pulsation model construction unit 22 of the reference pulsation model management unit 20 in FIG. 2. The reference pulsation model construction unit 22 constructs (i.e., generates) the reference pulsation model G for each user by using a variety of information included in the user information DB 21. The reference pulsation model construction unit 22 includes a data inspection unit 220 for determining whether data stored in the user information DB 21 should be used for the reference pulsation model G or not and a model calculation unit 226 for calculating the reference pulsation model G, as modeled reference pulsation information in regard to each user, from the pulsation information (e.g., cardiac activity information A1u or pulsation interval information calculated from the cardiac activity information A1u) included in the user information DB 21.

The data inspection unit 220 includes a cardiac activity information inspection unit 221 for inspecting the quality or property of the pulsation information indicating the cardiac activity (e.g., the cardiac activity information A1u or the pulsation interval information calculated from the cardiac activity information A1u), a situation determination unit 222 that determines an environmental situation in which the user is placed at the time of measuring the cardiac activity information A1u (e.g., environment information B0u), a user action determination unit 223 that determines the user's action at the time of measuring the cardiac activity information A1 (e.g., user action information A2u), and a data usage determination unit 224 that determines whether the cardiac activity information A1u should be used for the construction of the reference pulsation model G or not based on the result of the three inspections/determinations described above.

When the reference pulsation model G is calculated by using the whole of the user information stored in the user information DB 21, there is a possibility that the reference pulsation model G cannot be constructed appropriately if a problem has occurred in the quality of data. For example, data in which the pulsation interval is disturbed by the user's body motion is not suitable as the reference value H, and thus is desired to be excluded. The data inspection unit 220 makes it possible to construct (i.e., model) the reference pulsation model G of the user more appropriately by performing the data inspection in consideration of the quality of the cardiac activity information A1u itself, the user's action due to a body motion or the like, and the environment information B0 indicating the environment around the user and selecting the data to be used for the construction of the reference pulsation model G as the need arises.

The cardiac activity information inspection unit 221 first inspects the data quality of the cardiac activity information A1u itself. To take the pulse wave being the cardiac activity information A1u as an example, the cardiac activity information inspection unit 221 performs inspection regarding whether or not the pulsation interval can be obtained from the waveform of the pulse wave (e.g., whether or not appropriate peaks have appeared in the waveform), whether or not the pulsation interval obtained from the waveform of the pulse wave is in a range of values that a human can take on, or the like.

When it is determined that there is no problem in the quality of the cardiac activity information A1u (i.e., the cardiac activity information A1u satisfies a predetermined condition), the cardiac activity information inspection unit 221 inspects the property of the inputted cardiac activity information A1u. Since the reference pulsation model G is a model representing the reference pulsation of the user, an inspection process regarding the property of the cardiac activity information A1u exists so that the cardiac activity information A1u in a singular state such as a sudden change in the physical condition will not be used for the construction of the reference pulsation model G. Specifically, based on a variety of biological index obtained from the cardiac activity information A1u, it is determined whether the biological index is not a singular value indicating a singular state. Further, in this inspection process, it is possible either to use a raw signal of the cardiac activity information A1u or to calculate an index by using the pulsation interval information obtained from the cardiac activity information A1u. For example, a biological index such as an SDNN (standard deviation of the RR interval), a CVNN (i.e., SDNN/meanNN, where meanNN is the mean of the RR interval), an RMSSD (root mean square of differences between RR intervals adjacent to each other), an LF or an HF obtained from the pulsation interval is calculated, and based on such information, it is determined whether the input data is not singular data. The HF as a stress index is an acronym of high frequency (High Frequency), and means a fluctuating wave whose signal source is respiration having a cycle of approximately 3 to 4 seconds, or the sum total of power spectra in the frequency domain. The LF as a stress index is an acronym of low frequency (Low Frequency), and means a fluctuating wave called a Mayer wave whose signal source is blood pressure variation at a cycle of approximately 10 seconds, or the sum total of power spectra in the frequency domain.

When a change in a biological index at the time of a sudden change in the physical condition as the target of the detection is previously known, the determination in the inspection process may be made by utilizing such information. It is possible either to execute this inspection process by using a manually set threshold value or to make the determination automatically by using a technique such as machine learning. The cardiac activity information inspection unit 221 determines whether the inputted cardiac activity information A1u should be used for the construction of the reference pulsation model G or not by inspecting the quality and property of the cardiac activity information A1u included in the user information DB 21 by these processes.

The situation determination unit 222 determines external condition at the time point when the cardiac activity information A1u stored in the user information DB 21 is measured and determines whether the cardiac activity information A1u should be used for the construction of the reference pulsation model G or not depending on the external condition. For the determination of the external condition, the environment information B0u stored in the user information DB 21 is used. The environment information B0u is information that can be linked with the cardiac activity information A1u in a time series.

When the physical condition detection device 1 is supposed to be used in an automobile, traveling condition of the vehicle is as an example of the external condition as the object of the determination. Examples of the traveling condition include “whether or not the user as the subject is driving the vehicle”, “whether or not the user is performing a driving operation of reverse parking”, “whether the road on which the vehicle is traveling is an ordinary road or an expressway”, “whether the road on which the vehicle is traveling is congested”, and so forth. An in-vehicle environment is as an example of the external condition as the object of the determination. “Temperature in the vehicle”, “humidity in the vehicle” and so forth can be taken as examples of the in-vehicle environment. The situation determination unit 222 determines whether or not the data is acquired in an appropriate situation as data to be used for the construction of the reference pulsation model G. Thanks to this process, it is possible, for example, not to use the cardiac activity information A1u measured “in the driving operation of reverse parking” when the action of the driver as the subject is supposed to be significant. Incidentally, the method of determining the situation in this process is not limited to the method in the above-described example. The determination by the situation determination unit 222 may be made either based on the environment information B0u according to a previously set rule or by using a machine learning model.

The user action determination unit 223 determines the user's motion or action when the cardiac activity information A1u is measured. If the user's body motion is significant when the cardiac activity information A1u is measured, a situation where the information contains noise or the information is not measured appropriately is likely to occur. If the cardiac activity information A1u acquired in such a situation is used for the construction of the reference pulsation model G, the reference pulsation information regarding the user cannot be represented appropriately. Therefore, the user action determination unit 223 executes a process like previously detecting a user action like a body motion causing the occurrence of noise and not using the cardiac activity information A1u acquired at that time if the body motion is significant (e.g., if the magnitude of the body motion exceeds a predetermined threshold value). The user action determination unit 223 determines the user action based on the user action information A2. Specifically, the user action determination unit 223 detects the motion of the user as the subject based on a camera video or motion capture data and calculates the magnitude of the motion. The user action determination unit 223 may also be configured to determine not only the magnitude of the motion of the user but also whether or not the user is performing a predetermined particular motion (i.e., a motion that can cause the occurrence of noise). Incidentally, the means for detecting the user's motion or action is not limited to the above-described example. Further, the detection and determination made by the user action determination unit 223 can also be detection and determination making use of a machine learning model.

Furthermore, the situation determination unit 222 and the user action determination unit 223 may also be configured as a unit integrated as one processing block. In this case, this processing block is capable of determining the external condition and the user action by using both of the environment information B0 and the user action information A2.

The data usage determination unit 224 makes a final determination on whether or not to use each input of the cardiac activity information A1u stored in the user information DB 21 for the construction of the reference pulsation model G based on the inspection result and the physical condition determination result F obtained by the cardiac activity information inspection unit 221, the situation determination unit 222 and the user action determination unit 223. The data usage determination unit 224 determines whether to use data or not based on a previously set rule.

First, the data usage determination unit 224 refers to the inspection result of the cardiac activity information inspection unit 221 and confirms that there is no problem in the quality of the cardiac activity information A1u (i.e., the quality satisfies a predetermined condition) and the cardiac activity information A1u is not a singular value. Subsequently, the data usage determination unit 224 refers to the determination result of the situation determination unit 222 and confirms that the determined situation is not a previously set “situation corresponding to not using data”. Finally, the data usage determination unit 224 refers to the determination result of the user action determination unit 223 and confirms that the body motion is within a previously set threshold value and the body motion is not a previously set “action corresponding to not using data”. The data usage determination unit 224 stores these pieces of data, in which all of the inspection/determination results satisfy the previously set conditions, in a pulsation interval buffer 225 as data to be used for the construction of the reference pulsation model G.

Concrete information stored in the pulsation interval buffer 225 is, for example, time-series information on the pulsation interval obtained from the cardiac activity information A1u. The heat rate, the pulse rate, the RRI, the pulse interval, and so forth can be taken as examples of the pulsation interval. Further, this process is not executed in a lump for all of the data in the user information DB 21 but executed while segmenting the data according to specifications of data input to the physical condition detection unit 10. When the input specifications to the physical condition detection unit 10 specify a data length of 60 seconds and an input frequency of every 5 seconds, the data stored in the user information DB 21 is extracted successively in a form like that shown in FIG. 3 and the determination on whether to use the data or not in regard to the cardiac activity information A1u, the environment information B0 and the user action information A2 each for 60 seconds is carried out while temporally shifting the data.

While the data inspection unit 220 includes the cardiac activity information inspection unit 221, the situation determination unit 222 and the user action determination unit 223 in order to inspect each item of information, the data inspection unit 220 may also be configured while excluding part of these units. For example, the data inspection unit 220 may include only the cardiac activity information inspection unit 221 and the situation determination unit 222, or include only the cardiac activity information inspection unit 221. The configuration of the data inspection unit 220 can be modified depending on the information acquired as the user information.

The model calculation unit 226 calculates the reference pulsation model G for each user by using the inspected pulsation interval time-series information stored in the pulsation interval buffer 225 and stores the calculated reference pulsation model G in the reference pulsation model DB 23. First, an example of calculating the reference pulsation model representing the RR interval of each user by using the RR interval as the pulsation interval information will be shown below. For example, information in which the RR intervals are arranged in a time series has been stored in the pulsation interval buffer 225.

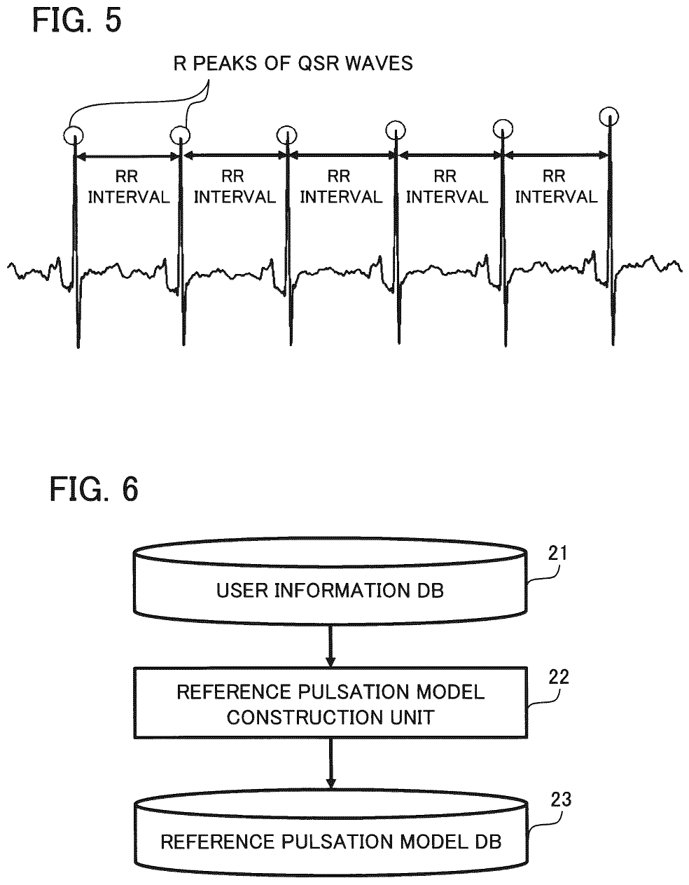

FIG. 5 is a diagram showing an example of the RR intervals as information calculated from an electrocardiogram. Here, an example of constructing the reference pulsation model G by using the Gaussian distribution (normal distribution) will be shown. Expression (1) represents the reference pulsation model G when the distribution of the pulsation interval is assumed to be the normal distribution. In the expression (1), X represents a stochastic variable, μ_{B} represents the mean value of the RR intervals stored in the pulsation interval buffer 225, σ_{B} represents the standard deviation of the RR intervals stored in the pulsation interval buffer 225, and N(μ_{B}, σ_{B}²) represents a one-dimensional normal distribution.

$$X ∼ N ( {μ}_{B} , {σ}_{B}^{2} ) \qquad ( 1 )$$

By stochastically modeling the reference pulsation of each user as above, it becomes possible to sample the reference value H corresponding to each user. While the modeling method of the reference pulsation model G in the case of assuming the Gaussian distribution is shown here, the modeling method is not limited to the above-described method. For example, the modeling method of the reference pulsation model G can also be a method using a Gaussian mixture model or a method using a neural network or the like. Further, the modeling method of the reference pulsation model G can also be a method using a non-parametric stochastic model obtained by probability density estimation or the like.

An example of calculating a model representing the RR interval as the reference pulsation model G has been presented above. However, the reference pulsation model G does not necessarily have to be a model representing a value indicating the pulsation interval itself such as the RR interval. The reference pulsation model G can also be a model targeted for a variety of index obtained from the pulsation interval, such as the SDNN, the CVNN, the RMSSD, the LF or HF, the mean value or median of the RR interval, pNN20 (ratio of values greater than or equal to 20 msec among differences between RR intervals adjacent to each other), or pNN50 (ratio of values greater than or equal to 50 msec among differences between RR intervals adjacent to each other). The reference pulsation model G in that case is a model representing the reference value H of the index obtained from the pulsation interval. Further, it is also possible to store both of the reference pulsation model G calculated from the RR interval and the reference pulsation model G obtained from a variety of index as the reference pulsation models G in the form of a database.

### Reference Pulsation Model DB 23

The reference pulsation model DB 23 is an area for storing the reference pulsation model for each user calculated by the reference pulsation model construction unit 22. In the reference pulsation model DB 23, each reference pulsation model is linked with a user ID, and data is stored in a format in which a reference pulsation model G can be referred to by using a user ID.

### Reference Pulsation Calculation Unit 24

The reference pulsation calculation unit 24 receives a user ID as an input and calculates the reference pulsation of the user. An example of a process of calculating the reference pulsation will be described below. First, the reference pulsation calculation unit 24 extracts the reference pulsation model G linked with the user ID from the reference pulsation model DB 23 by using the user ID. Subsequently, the reference pulsation calculation unit 24 samples an arbitrary number of reference values H from the extracted reference pulsation model G. Subsequently, the reference pulsation calculation unit 24 obtains a representative value from the sampled reference values H and outputs the obtained representative value as a final reference value H. A mean value, a median or the like can be used as the representative value. In cases where Gaussian distribution is used as the reference pulsation model G, the reference pulsation calculation unit 24 may directly output the mean value of the Gaussian distribution as the final reference value H of the reference pulsation model G.

It is also possible for the reference pulsation calculation unit 24 to output a plurality of candidates for the reference value H as the reference value H without using the representative value. The method of using the reference value H in that case will be described later in an item of the normalized feature extraction unit 13 in the physical condition detection unit 10.

### Process in Reference Pulsation Model Management Unit 20

FIG. 6 is a diagram showing a general outline of a process flow executed by the reference pulsation model construction unit 22 of the reference pulsation model management unit 20 in FIG. 2. As shown in FIG. 6, the reference pulsation model management unit 20 receives previously collected information in the user information DB 21 as an input and repeats the inspection of data by the data inspection unit 220 until all of the data regarding the user as the target are referred to. By this process, data for forming the reference pulsation model G by use of the data are stored in the pulsation interval buffer 225. After the inspection of all of the data is completed, the reference pulsation calculation unit 24 obtains the reference pulsation model G and stores the reference pulsation model G in the reference pulsation model DB 23 in the form of having been linked with the user ID.

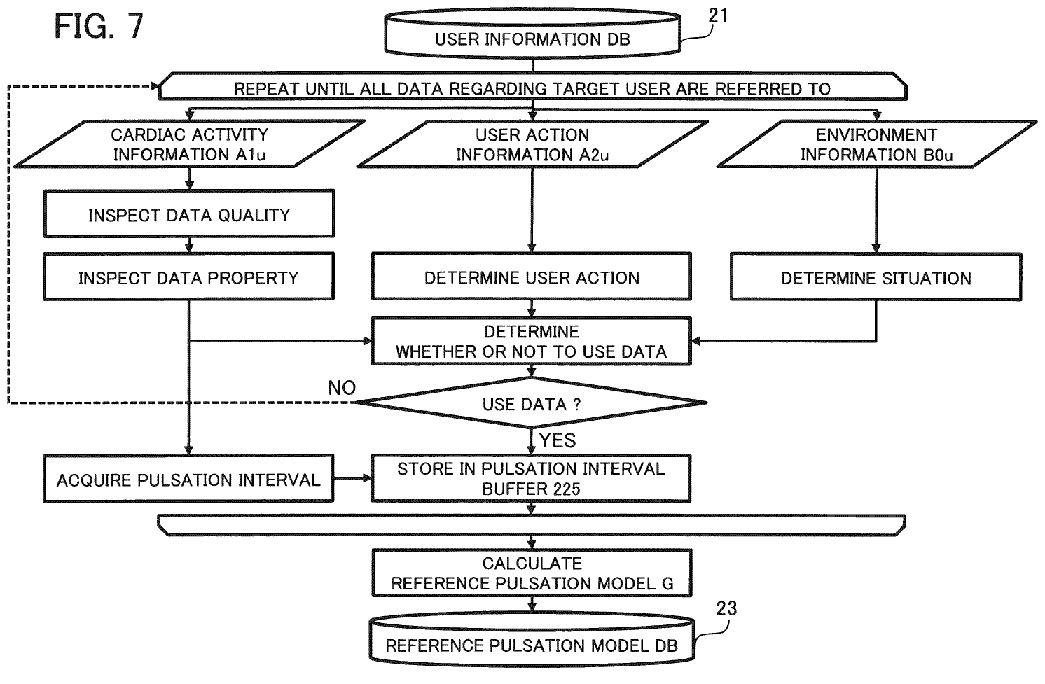

FIG. 7 is a diagram showing details of the process flow executed by the reference pulsation model construction unit 22 of the reference pulsation model management unit 20 in FIG. 2. The process of the reference pulsation calculation unit 24 is not included in the process flow in FIG. 7 since the process is used from the physical condition detection unit 10. As shown in FIG. 7, the reference pulsation model management unit 20 receives the cardiac activity information A1u, the user action information A2 and the environment information B0, which are the previously collected information in the user information DB 21, as the input and repeats the inspection of data by the data inspection unit 220 until all of the data regarding the user as the target are referred to. The data inspection unit 220 of the reference pulsation model management unit 20 inspects the data quality and inspects the data property in regard to each piece of the cardiac activity information A1u and determines whether data should be used or not in regard to each piece of data in the cardiac activity information A1u. The data inspection unit 220 makes a determination in regard to each user action in regard to each piece of the cardiac activity information A1u and determines whether data should be used or not in regard to each piece of data of the user action. The data inspection unit 220 makes a determination in regard to each situation indicated by the environment information B0 and determines whether data should be used or not in regard to each piece of data in the environment information B0. When data should be used, the data inspection unit 220 stores the data for forming the reference pulsation model G in the pulsation interval buffer 225. After the inspection of all of the data is completed, the reference pulsation calculation unit 24 obtains the reference pulsation model G and stores the reference pulsation model G in the reference pulsation model DB 23 in the form of having been linked with the user ID.

The reference pulsation model management unit 20 manages the reference pulsation information in regard to each user using the physical condition detection unit 10 as the subsequent stage, and is capable of acquiring the reference pulsation of each user at any time.

### (1-4) Physical Condition Detection Unit 10

The physical condition detection unit 10 detects the presence/absence of a change in the physical condition (e.g., a sudden change in the physical condition, namely, a sudden deterioration in the physical condition) by using the user's cardiac activity information A1 acquired in real time. The physical condition detection unit 10 includes the process continuation determination unit 11 that inspects the quality of the cardiac activity information A1, the situation the user is in, and the user's action based on the sensor information A0 and the environment information B0 inputted thereto and determines whether the physical condition detection process should be continued or not, the pulsation interval acquisition unit 12 that acquires the pulsation interval from the cardiac activity information A1, the normalized feature extraction unit 13 that calculates a feature value to be used for detecting a sudden change in the physical condition, in which the individual difference has been corrected, from the time series of the acquired pulsation intervals C, and the physical condition determination unit 14 that determines the presence/absence of a sudden change in the physical condition by using the corrected feature value.

### Process Continuation Determination Unit 1

The process continuation determination unit 11 determines whether the measured cardiac activity information A1 is information appropriate as the input to the physical condition detection unit 10 or not by using the sensor information A0 and the environment information B0. For example, when the cardiac activity information A1 has not been successfully acquired in sufficiently high quality due to the user's intense body motion at the time of the sensing by the sensor 103 (FIG. 1), a reliable result cannot be obtained even if the detection of a sudden change in the physical condition is carried out by using the information. In order to prevent such cases, before the detection of a sudden change in the physical condition is carried out, the process continuation determination unit 11 determines whether the process of detecting a sudden change in the physical condition should be continued with the measured cardiac activity information A1 or not by using the measured cardiac activity information A1.

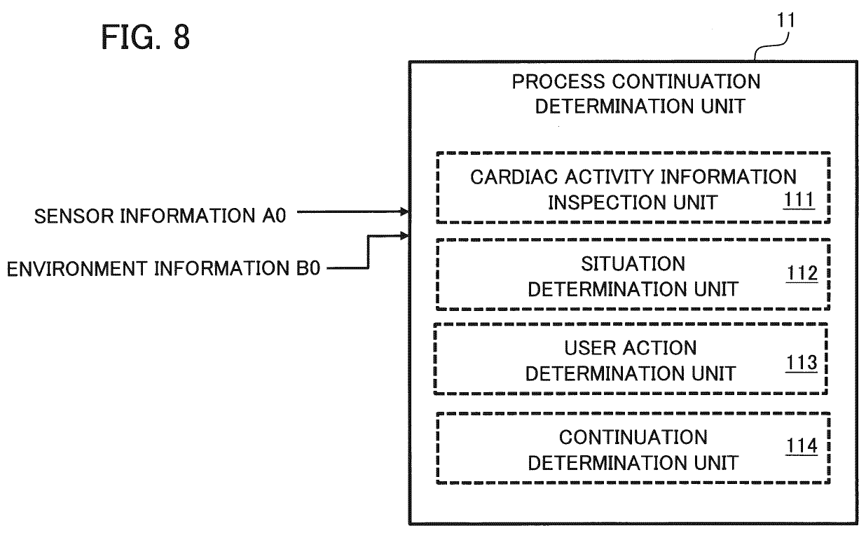

FIG. 8 is a block diagram showing the functional configuration of the process continuation determination unit 11 of the physical condition detection unit 10 in FIG. 2. In the first embodiment, the sensor information A0 includes the cardiac activity information A1 and the user action information A2. The process continuation determination unit 11 includes a cardiac activity information inspection unit 111, a situation determination unit 112, a user action determination unit 113 and a continuation determination unit 114. The process continuation determination unit 11 has a configuration similar to that of the data inspection unit 220 of the reference pulsation model construction unit 22 in the reference pulsation model management unit 20 shown in FIG. 4 and executes a similar process. The process continuation determination unit 11 differs from the data inspection unit 220 of the reference pulsation model construction unit 22 in that the input is not the information from the user information DB 21 but the sensor information A0 and the environment information B0 acquired in real time. The continuation determination unit 114 in the process continuation determination unit 11 has a function similar to that of the data usage determination unit 224 in the data inspection unit 220.

The process continuation determination unit 11 does not necessarily have to include all of the cardiac activity information inspection unit 111, the situation determination unit 112 and the user action determination unit 113 but can also be formed with one of these units or a combination of two of these units. The process continuation determination unit 11 is capable of changing its process to be executed depending on the information inputted to the physical condition detection unit 10. For example, in cases where it is difficult for the physical condition detection unit 10 to acquire the environment information B0 in real time, the process continuation determination unit 11 may be configured to include only the cardiac activity information inspection unit 111 and the user action determination unit 113.

### Pulsation Interval Acquisition Unit 12

The pulsation interval acquisition unit 12 receives the cardiac activity information A1 as the input and extracts the pulsation interval C from the time-series signal of the electrocardiogram waveform. When the electrocardiogram waveform shown in FIG. 5 is used as the input, the pulsation interval acquisition unit 12 acquires the RR interval from the time-series signal of the electrocardiogram waveform. However, the method of acquiring the pulsation interval C is not limited to the method of acquiring the pulsation interval C from the electrocardiogram waveform. The method of acquiring the pulsation interval C can be, for example, a method of detecting peaks of the waveform by peak detection algorithm and thereafter calculating the interval between the peaks, or a method of directly calculating the pulsation interval C from the waveform by using machine learning algorithm such as neural network. Further, the input can be any type of information from which the pulsation interval C of the heart can be acquired, and can also be different information (e.g., pulse wave waveform or the like) as long as the pulsation interval C of the heart can be acquired from the information.

### Normalized Feature Extraction Unit 13

The normalized feature extraction unit 13 calculates the feature value to be used for detecting a change in the physical condition (e.g., a sudden change in the physical condition, namely, a sudden deterioration in the physical condition) by using the time-series information on the pulsation interval C acquired from the pulsation interval acquisition unit 12 as an input. Further, the normalized feature extraction unit 13 not only simply extracts the feature value but also extracts the feature value in which the individual difference has been corrected, by converting the feature value by using the reference value H of each user acquired from the reference pulsation calculation unit 24. The feature value handled in the present application is an index (e.g., value) obtained by performing a process on the time-series information on the pulsation interval C. The SDNN, the CVNN, the pNN20, the pNN50, the RMSSD, entropy, multiscale entropy, the LF or HF, the mean value or median of the pulsation interval C, and so forth can be taken as examples of the feature value. The feature value handled by the normalized feature extraction unit 13 is not limited to the above-described values, and is an index (e.g., value) obtained by performing a certain predetermined process on the pulsation interval C.

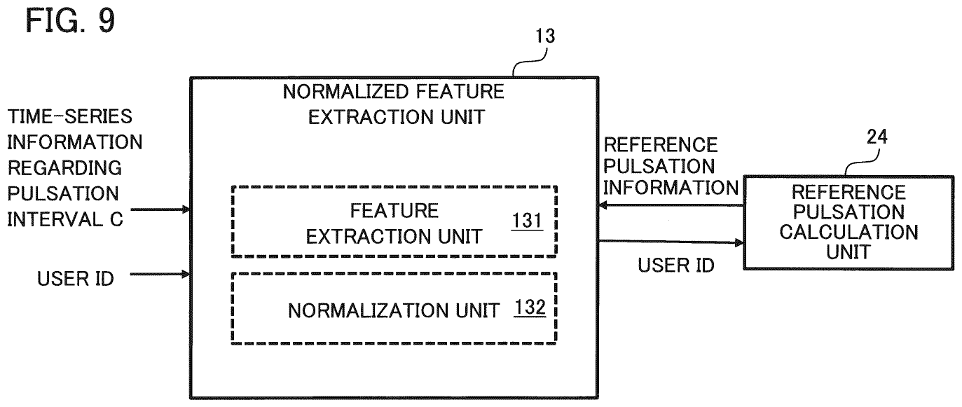

FIG. 9 is a block diagram showing the functional configuration of the normalized feature extraction unit 13 of the physical condition detection unit 10 in FIG. 2. The normalized feature extraction unit 13 includes a feature extraction unit 131 that calculates a variety of feature value from the pulsation interval C and a normalization unit 132 that normalizes the calculated feature value by using the reference pulsation information regarding each user acquired from the reference pulsation calculation unit 24. The feature extraction unit 131 calculates the variety of feature value by performing the above-described feature extraction process on the inputted time-series information on the pulsation interval C. The feature value calculated can be either one feature value or two or more feature values as needed.

Subsequently, the normalization unit 132 corrects the individual difference in the variety of feature value calculated from the pulsation interval C by normalizing the feature value acquired from the feature extraction unit 131 by using the reference pulsation information regarding each user. The normalization unit 132 first inputs the user ID to the reference pulsation calculation unit 24 and thereby acquires the reference pulsation information identified by the user ID. The reference pulsation information acquired here is the reference value H of the feature value (index) extracted by the feature extraction unit 131. That is, the reference pulsation model management unit 20 needs to previously construct the reference pulsation model G for previously calculating the reference value H of the feature value to be used by the normalized feature extraction unit 13. As another method of acquiring the reference value H, there is a method in which an arbitrary number of reference pulsations (e.g., RR intervals or the like) are obtained by the reference pulsation calculation unit 24 and a feature value acquired by performing the feature extraction process on a reference pulsation set made up of a plurality of reference pulsations obtained by the reference pulsation calculation unit 24 is acquired as the reference value H. However, in cases where the feature value as the target is a value having taken the time series of the pulsation interval C into account, the reference pulsation model G also needs to be formed with a time-series model. The reference value H of the variety of feature value acquired by the processing so far can be regarded as an index representing a biological property of the user using the physical condition detection unit 10 at the time with no sudden change in the physical condition (i.e., in the non-paroxysmal time). The physical condition detection unit 10 corrects the feature value calculated by the feature extraction unit 131 by using this reference value H.

Examples of the method of the correction include a method of taking the difference between the feature value obtained by the feature extraction unit 131 and the reference value H, a method of using a value obtained by dividing the feature value obtained by the feature extraction unit 131 by the reference value H, and so forth. Irrespective of which method is used, an index representing variation of the feature value of each user from the reference value H is used for the correction of the feature value. The feature in which the individual difference of each user has been corrected can be obtained by excluding a component of the reference value H of each user from the feature value obtained by the feature extraction unit 131.

While the above description has presented an example in which one reference value H is acquired and the normalization process is executed by using the value when normalizing each feature value, the processing by the normalization unit 132 is not limited to the above-described processing. Further, it is also possible to perform the normalization process on one feature value based on a plurality of reference value candidates without performing the normalization process on one feature value by using one reference value H. The processing in this case is executed as follows. The following description will be given of a normalization process taking the difference between the reference value H and the feature value.

First, the normalized feature extraction unit 13 acquires N reference value candidates from the reference pulsation calculation unit 24. Subsequently, the normalized feature extraction unit 13 takes the difference between the feature value obtained by the feature extraction unit 131 and each reference value candidate. At this stage, N difference values are obtained. Finally, the normalized feature extraction unit 13 takes the mean value or median of the N difference values and employs the obtained value as the normalized feature value. Since it is known that fluctuation occurs to information regarding the pulsation interval C even when the same subject and the time with no sudden change in the physical condition are assumed, it is difficult to represent the information as the reference by one value. By executing the feature value normalization process based on a plurality of reference value candidates obtained from the reference pulsation model G by this processing, the feature from which the influence of the fluctuation occurring to the reference value H has been excluded can be obtained.

### Physical Condition Determination Unit 14

The physical condition determination unit 14 determines the physical condition (e.g., the presence/absence of a sudden change in the physical condition) of the user identified by the user ID by using the feature value calculated by the normalized feature extraction unit 13. The physical condition determination result F may be configured to be represented as a value being one of two values like “0” and “1” or a decimal fraction between 0 and 1 as a value indicating the probability that a sudden change in the physical condition has occurred. The physical condition determination result F may also be configured to indicate the determination while making discrimination among types of the sudden change in the physical condition. In that case, as many physical condition determination results F as the number of pertinent types of the sudden change in the physical condition are outputted. Further, the method of the determination can be a determination method using a threshold value, a determination method using machine learning, or the like.

The determination method using a threshold value will be described below. The physical condition determination unit 14 determines that there is a sudden change in the physical condition when the value of the normalized feature value exceeds a previously set threshold value or falls below a previously set threshold value. When a plurality of feature values are handled, a method in which a threshold value is set in regard to each feature value and a method of making the determination on a sudden change in the physical condition according to an arbitrarily set rule by referring to the values of the plurality of feature values can be taken as examples of the determination method. The setting of the threshold value may be either manually made or automatically calculated based on a normalized feature value obtained from previously collected cardiac activity information A1u. Further, since the normalized feature value is an index in which the individual difference has been corrected, it is not necessarily essential at the time of setting the threshold value to use the cardiac activity information A1u on the user using the physical condition detection by the physical condition detection device 1. The physical condition determination unit 14 may also set the threshold value based on a normalized feature value obtained from the cardiac activity information A1u measured in a different user group. It is also possible for the physical condition determination unit 14 to set a common threshold value for the physical condition detection unit 10 based on normalized feature values previously calculated for a plurality of users and thereafter adjust the threshold value by using the information on the user using the physical condition detection unit 10.

The determination using machine learning will be described below. The physical condition determination unit 14 determines the presence/absence of a sudden change in the physical condition by inputting the feature value to a previously learned machine learning model. At the time of forming the machine learning model, it is also possible to learn the machine learning model based on normalized feature values obtained from a user group other than the user using the physical condition detection unit 10 similarly to the above-described threshold value setting method. Further, it is also possible to employ a method of learning a machine learning model common to the physical condition detection unit 10 based on normalized feature values previously calculated for a user group other than the user using the physical condition detection unit 10 and thereafter fine-tuning the machine learning model by using the information on the user using the physical condition detection unit 10. The algorithm of the machine learning used here can be algorithm of any type. A learning model by the neural network, the Gaussian mixture model (GMM), a class classification model such as SVM (Support Vector Machine), and a learning model based on decision tree such as XGBoost can be taken as examples of usable algorithm.

### Process by Physical Condition Detection Unit 10

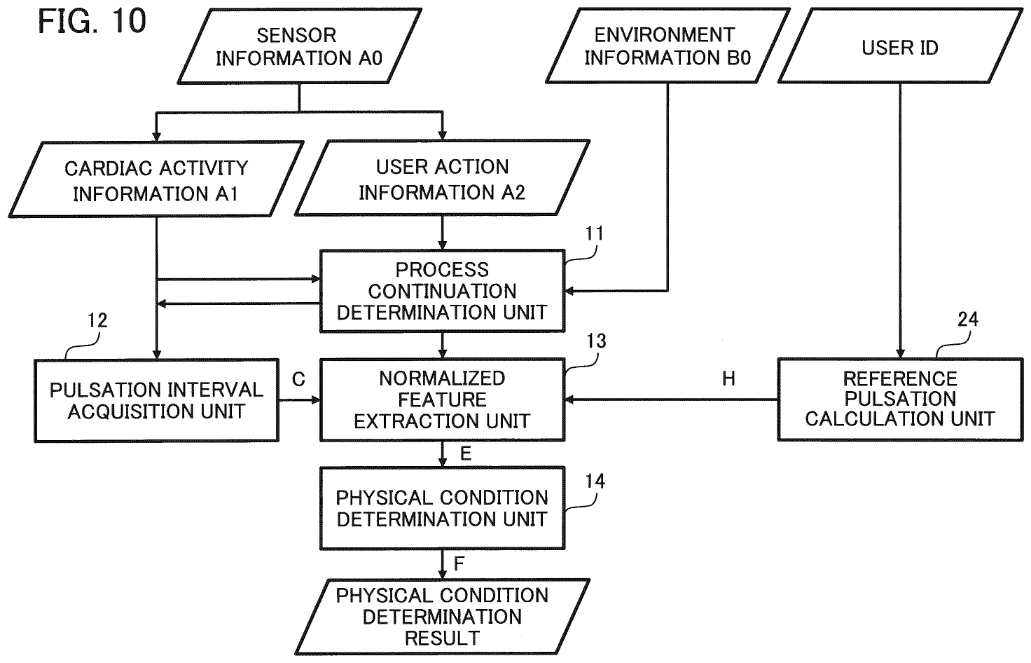

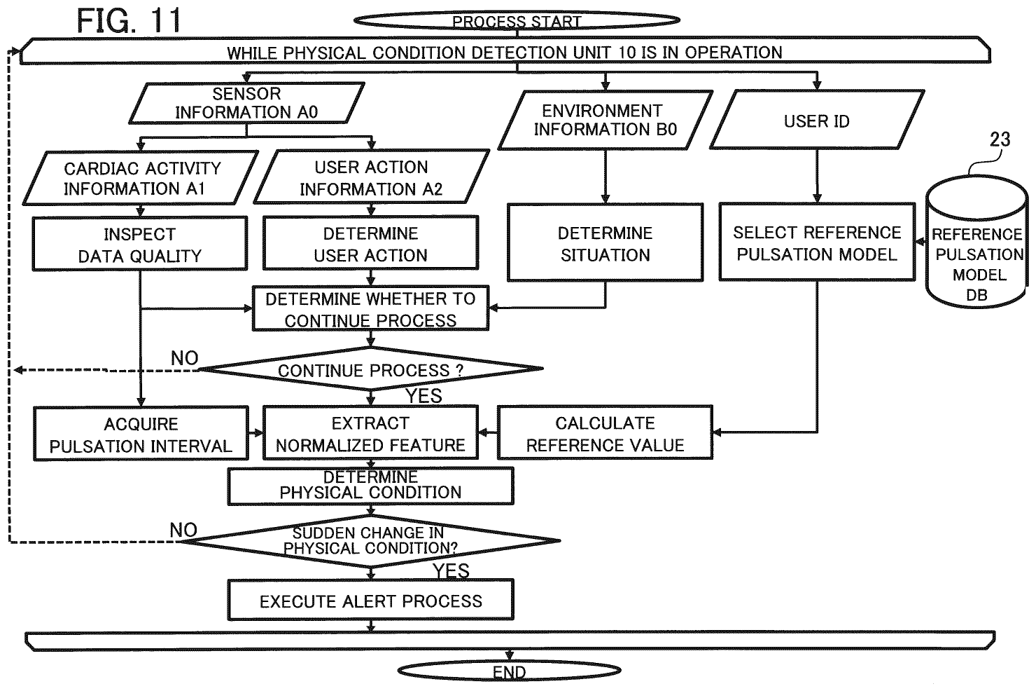

FIG. 10 is a diagram showing a general outline of a process flow executed by the physical condition detection unit 10 in FIG. 2. FIG. 11 is a diagram showing details of the process flow executed by the physical condition detection unit 10 in FIG. 2. The inputs to the physical condition detection unit 10 are the sensor information A0 including the cardiac activity information A1 and the user action information A2, the environment information B0 to be used for determining the situation the user is in, and the user ID for identifying the user using the physical condition detection unit 10. The output from the physical condition detection unit 10 is the physical condition determination result F regarding the physical condition (e.g., physical condition determination result F regarding a sudden change in the physical condition). Further, in the first embodiment, the input of the environment information B0 is not necessarily essential, in which case the environment information B0 and the situation determination process using the environment information B0 as the input in FIG. 11 can be left out.

First, the physical condition detection unit 10 determines whether the process of detecting a sudden change in the physical condition should be continued or not based on the cardiac activity information A1 and the environment information B0. When it is determined that the detection process should not be continued, the physical condition detection unit 10 waits for the next input without executing the subsequent processing. When it is determined that the detection process should be continued, the physical condition detection unit 10 makes the pulsation interval acquisition unit 12 acquire the pulsation interval C from the cardiac activity information A1. The normalized feature is extracted from the acquired pulsation interval C by the normalized feature extraction unit 13, and the physical condition determination result F is outputted by the physical condition determination unit 14. When it is determined that there is a sudden change in the physical condition, an alert process corresponding to the physical condition detection device is executed. As examples of the alert process, there are a process of displaying a warning on the display device 106 (FIG. 1), a process of outputting a warning sound from a speaker (not shown), a process of making a vehicle autonomously move to a safe place and stop in cases of a vehicle performing autonomous driving, and so forth. The above-described processing is repeated until the process by the physical condition detection device 1 ends.

### (1-5) Effect

As described above, according to the first embodiment, the corrected feature value E is generated by correcting the influence of the individual difference in the pulsation information on the feature value, and the determination on a change in the physical condition (e.g., a sudden change in the physical condition, namely, a sudden deterioration in the physical condition) is made based on the corrected feature value E, and thus physical condition detection robust to the individual difference in the pulsation interval C can be realized.

### (2) Second Embodiment

In a second embodiment, a description will be given of a physical condition detection device, a physical condition detection system, a physical condition detection method and a physical condition detection program used for detecting a change in the physical condition (especially, a sudden change in the physical condition, namely, a sudden deterioration in the physical condition) of the user by using a corrected feature value E obtained by correcting the influence of the individual difference in the pulsation interval on the feature value and an influence of a change in the situation the user is in on the feature value.

### (2-1) Physical Condition Detection System

The HW configuration of the physical condition detection system according to the second embodiment is similar to that in the first embodiment (FIG. 1). The physical condition detection system according to the second embodiment includes a physical condition detection device 1a and the sensor 103. The physical condition detection system according to the second embodiment may include the input device 104, the storage 105 and the display device 106. The physical condition detection device 1a is a device capable of executing a physical condition detection method according to the second embodiment. Alternatively, the physical condition detection device 1a is a computer capable of executing a physical condition detection program according to the second embodiment.

### (2-2) Physical Condition Detection Device 1a

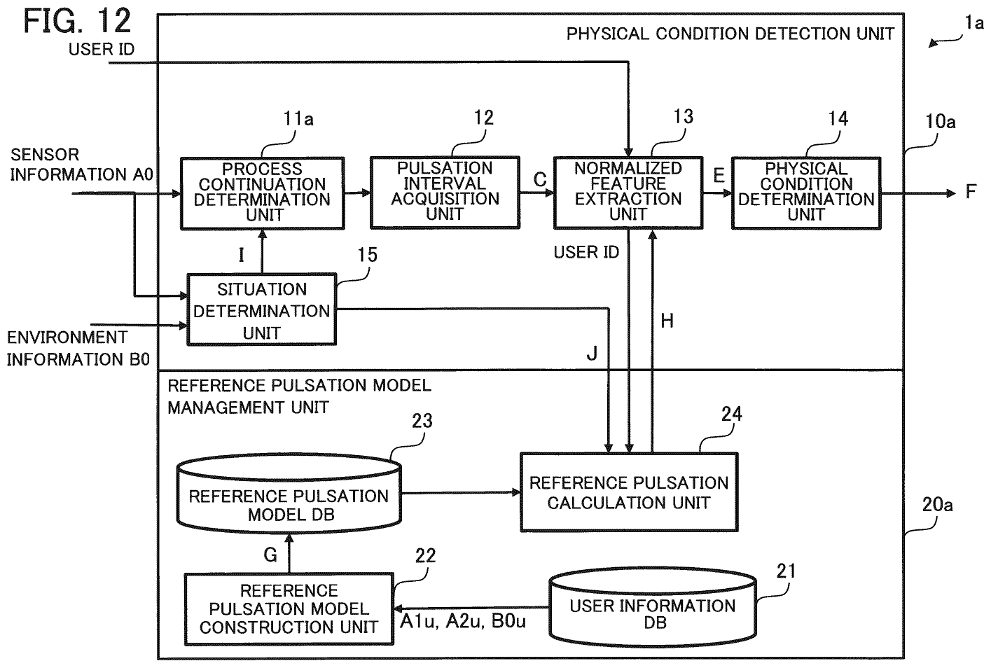

FIG. 12 is a block diagram showing the functional configuration of the physical condition detection device 1a according to the second embodiment. Similarly to the physical condition detection device 1 according to the first embodiment, the physical condition detection device 1a according to the second embodiment includes a reference pulsation model management unit 20a for acquiring the reference information regarding each user as a subject and a physical condition detection unit 10a that receives the pulsation information acquired from the sensor 103 and the environment information B0 indicating the environment surrounding the user as the subject as inputs and detects a change in the physical condition (e.g., a sudden change in the physical condition, namely, a sudden deterioration in the physical condition) of the user.

The reference pulsation model management unit 20a includes the user information DB 21, the reference pulsation model construction unit 22, the reference pulsation model DB 23 and the reference pulsation calculation unit 24. The physical condition detection unit 10a includes a process continuation determination unit 11a, the pulsation interval acquisition unit 12, the normalized feature extraction unit 13, the physical condition determination unit 14 and a situation determination unit 15.

The physical condition detection device 1a according to the second embodiment differs from the physical condition detection device 1 according to the first embodiment in that the physical condition detection device 1a is configured to calculate a reference value H corresponding to the situation the user is in from the reference pulsation model G, the situation determination unit 15 is independent of the process continuation determination unit 11a, and a situation determination result J is used for the calculation of the reference value H. By using the physical condition detection device 1a according to the second embodiment, the corrected feature value E corrected appropriately can be obtained by making not only the correction of the influence of the individual difference in the pulsation interval C on the feature value but also the correction regarding the influence of a change in the situation the user is in on the feature value.

### (2-3) Reference Pulsation Model Management Unit 20a

The reference pulsation model management unit 20a differs from the reference pulsation model management unit 20 in the first embodiment in the construction method of the reference pulsation model G and the calculation method of the reference pulsation. Except for the above-described features, the configuration of the reference pulsation model management unit 20a is the same as that of the reference pulsation model management unit 20 in the first embodiment. Further, the user information in the second embodiment is also the same as that in the first embodiment.

### Reference Pulsation Model Construction Unit 22

The reference pulsation model construction unit 22 in the second embodiment not only constructs the reference pulsation model G by modeling the pulsation interval information as the reference of each user but also performs the modeling by making the correction also regarding the influence of the situation the user is in on the pulsation interval information. The description here will be given mainly of the difference from the reference pulsation model construction unit 22 in the first embodiment.

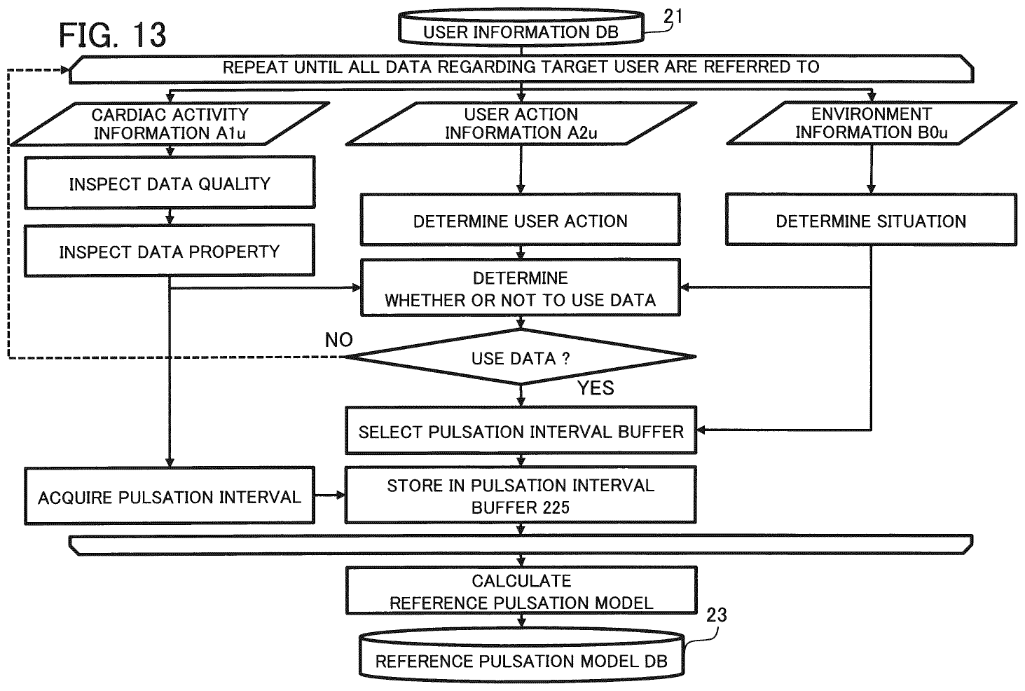

FIG. 13 is a diagram showing details of a process flow executed by the reference pulsation model construction unit 22 of the reference pulsation model management unit 20a in FIG. 12. The process in the second embodiment shown in FIG. 13 is the same as the process in the first embodiment in regard to the processing to the process of making the data inspection unit 220 execute the inspection of information by using the information regarding each user included in the user information DB 21 as the input. In the second embodiment, processing different from the processing in the first embodiment is executed from a process of storing data to be used for the construction of the reference pulsation model G in the pulsation interval buffer 225. In the second embodiment, as many buffers as the number of previously set situations are prepared and the pulsation interval is stored while switching the pulsation interval buffer 225 depending on the situation. When four situations: situation #1 to situation #4, have been set as situations assumed by the physical condition detection device, the pulsation interval measured in each situation is stored in a corresponding one of the four buffers.

The model calculation unit 226 constructs the reference pulsation model G by using the pulsation interval buffer 225 storing the pulsation interval in regard to each situation. As an example of the reference pulsation model G in the second embodiment, a model formed by the sum of the reference pulsation model G representing the reference value H and a situation difference model representing the influence of the situation on the pulsation interval will be shown below. The reference pulsation model G described here is synonymous with the reference pulsation model G in the first embodiment and is a model representing the pulsation information as the reference of each user. The situation difference model is a model representing the influence of the situation the user is in on the pulsation information, namely, variation in the pulsation interval information caused by a particular situation. Here, for the sake of explanation, a description will be given of an example in a case where each of the reference model and the situation difference model is represented by Gaussian distribution. Further, here, an example in which four situations: situation #1 to situation #4, have been set will be described.

First, a situation as the reference of all the situations is set from among the situations #1 to #4. As an example of the method of setting the reference, it is possible to consider a method of setting a situation whose time for which the user using the physical condition detection device la is placed in the situation is the longest as the reference. In the following description, the situation set by this process is referred to as a reference situation. Here, the description will be given of a case where the situation #1 has been set as the reference situation.

Subsequently, the model calculation unit 226 extracts the pulsation interval in the reference situation from the pulsation interval buffer 225 and calculates the reference model. The calculation method is the same as the calculation method used by the model calculation unit 226 in the first embodiment.

Subsequently, the model calculation unit 226 constructs the situation difference model by using the pulsation intervals respectively measured in the situations #2 to #4. The situation difference model is a model obtained by modeling the difference between the pulsation interval information in each situation and the mean value in the reference model in order to represent the variation in the pulsation interval when the situation changed from the reference situation to each of the other situations. When the reference model X_{B} is represented by expression (2), the difference between each pulsation interval in the situation #N (N: positive integer) and the reference can be represented by expression (3). Here, x_{N}⁽ⁱ⁾ represents the i-th pulsation interval in the situation #N and μ₁ represents the mean value in the reference model.

$${X}_{B} ∼ N ( {μ}_{1} , {σ}_{1}^{2} ) \qquad ( 2 )$$

$${x}_{N}^{( i )} ∼ {μ}_{1} \qquad ( 3 )$$

In this case, the situation difference model in the situation #N is represented by expression (4). Here, SN represents the situation difference model in the situation #N, μ_{diffN} represents the mean value of the difference, and σ_{diffN} represents the standard deviation of the difference.

$${S}_{N} ∼ N ( {μ}_{{diff}_{N}} , {σ}_{{diff}_{N}}^{2} ) \qquad ( 4 )$$

The reference pulsation model X in the situation #N is represented by the sum of the reference model X_{B} and the situation difference model S_{N} like expression (5).

$${X}_{N} ∼ {X}_{B} + {S}_{N} \qquad ( 5 )$$

The model calculation unit 226 stores the calculated reference pulsation model X_{N} in the reference pulsation model DB 23 in the form of having been linked with the user ID. While the construction method (i.e., modeling method) of the reference pulsation model in the case of assuming the Gaussian distribution has been shown here, the modeling method is not limited to the above-described method. For example, it is also possible to use a Gaussian mixture model, a neural network or the like as each of the reference model and the situation difference model. Further, it is also possible to use a non-parametric stochastic model obtained by probability density estimation or the like.

In the above description, an example of calculating a model representing the RR interval as the reference pulsation model G has been presented. However, the reference pulsation model G does not necessarily have to be a model representing a value indicating the pulsation interval itself such as the RR interval. The reference pulsation model G can also be a model targeted for a variety of index obtained from the pulsation interval, such as the SDNN, the CVNN, the RMSSD, the LF or HF, the mean value or median of the RR interval, the pNN20, or the pNN50. The reference pulsation model G in that case is a model representing the reference value H of the index obtained from the pulsation interval.

Further, the device may also be configured to hold both of the reference pulsation model G calculated from the RR interval and the reference pulsation model G obtained from a variety of index.

### Reference Pulsation Calculation Unit 24

The reference pulsation calculation unit 24 calculates the reference pulsation corresponding to the situation of each user by using the user ID and the situation the user is in as inputs. The process regarding the calculation of the reference pulsation is similar to that in the first embodiment. The reference pulsation calculation unit 24 in the second embodiment differs from that in the first embodiment in that the situation the user is in is added as an input.

The normalized feature extraction unit 13 is capable of extracting the corrected feature value E from which the influence of the individual difference in the pulsation interval C on the feature value and the influence of the situation the user is in on the feature value have been removed, by executing the normalization of the feature value by using the reference pulsation obtained by the reference pulsation calculation in the second embodiment.

### 2-4

The physical condition detection unit 10a detects a change (e.g., the presence/absence of a sudden change) in the physical condition by using the user's cardiac activity information A1 acquired in real time. Further, the physical condition detection unit 10a normalizes the feature value, for realizing the detection of a sudden change in the physical condition, by using the reference value H corresponding to the situation of each user based on the “situation the user is in” determined based on the environment information B0 acquired in real time. The physical condition detection unit 10a includes the process continuation determination unit 11a that inspects the quality of the cardiac activity information A1 and the action of the user based on the inputted sensor information A0 and determines whether the process should be continued or not, the situation determination unit 15 that determines the situation the user is in based on the environment information B0, the pulsation interval acquisition unit 12 that acquires the pulsation interval C from the cardiac activity information A1, the normalized feature extraction unit 13 that calculates the feature value to be used for detecting a sudden change in the physical condition, in a form after correcting the variation in the pulsation interval C due to the individual difference and a change in the situation, from the time series of the acquired pulsation intervals C, and the physical condition determination unit 14 that determines the presence/absence of a sudden change in the physical condition by using the obtained corrected feature value E. In the following, a description will be given of the physical condition detection unit 10a, mainly of features different from those in the first embodiment.

### Situation Determination Unit 15

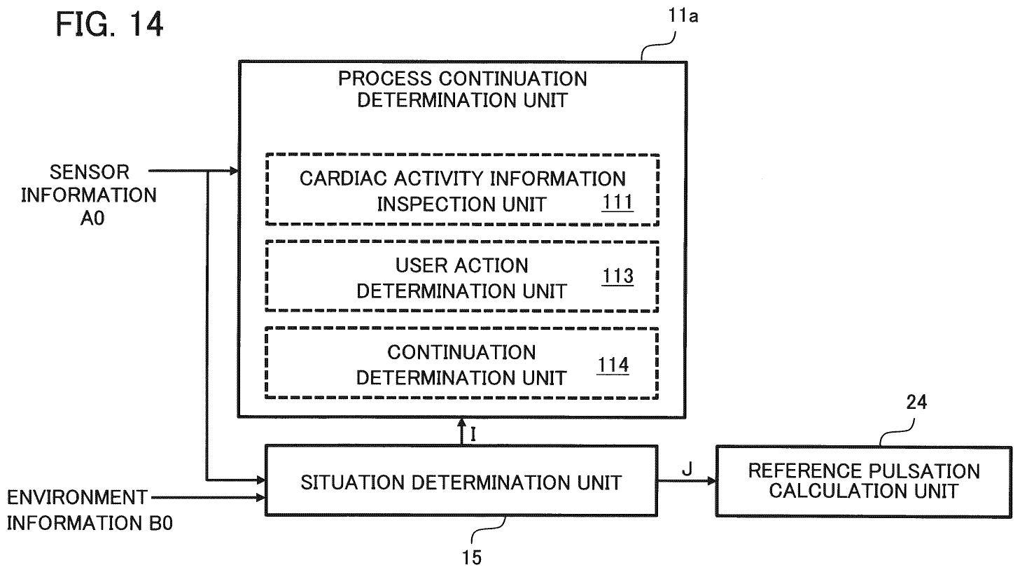

FIG. 14 is a block diagram showing the functional configuration of the process continuation determination unit 11a and the situation determination unit 15 of the physical condition detection unit 10a in FIG. 12. In FIG. 14, the situation determination unit 15 corresponds to the situation determination unit 112 in the process continuation determination unit 11 in the first embodiment, and is placed outside the process continuation determination unit 11a as an independent unit. The contents of the processing by each block are similar to those in the first embodiment. The situation determination unit 15 determines the situation the user is in by using the environment information B0 and the sensor information A0 as inputs, and inputs the result of the determination to the process continuation determination unit 11a. The physical condition detection unit 10a in the second embodiment differs from the physical condition detection unit 10 according to the first embodiment in that the result obtained by the situation determination unit 15 is used not only for the process continuation determination but also for the calculation of the reference pulsation.

### Processing in Physical Condition Detection Unit 10a

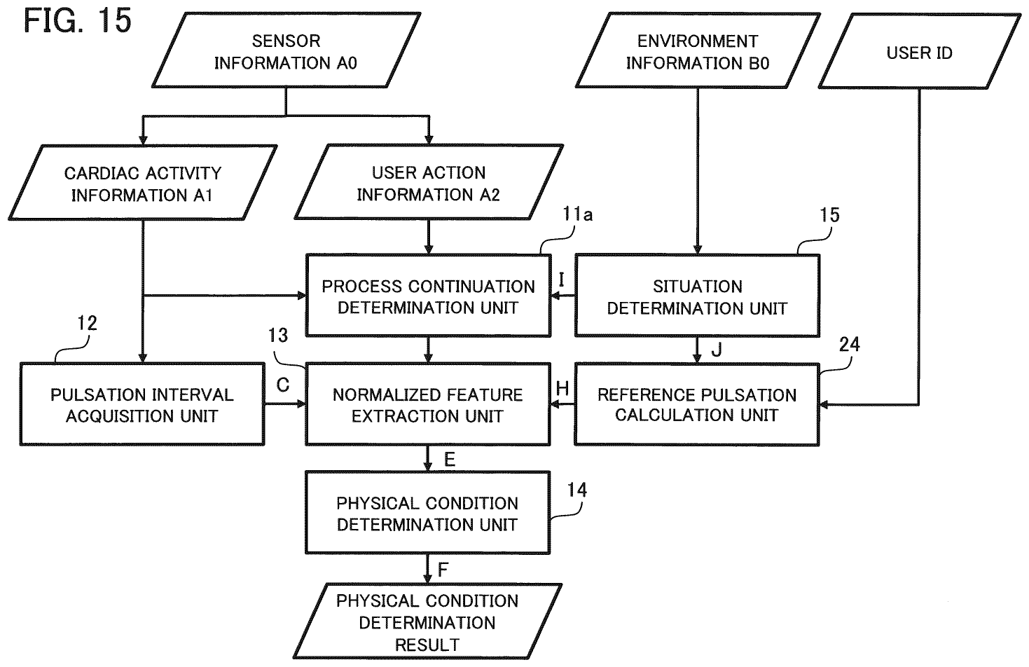

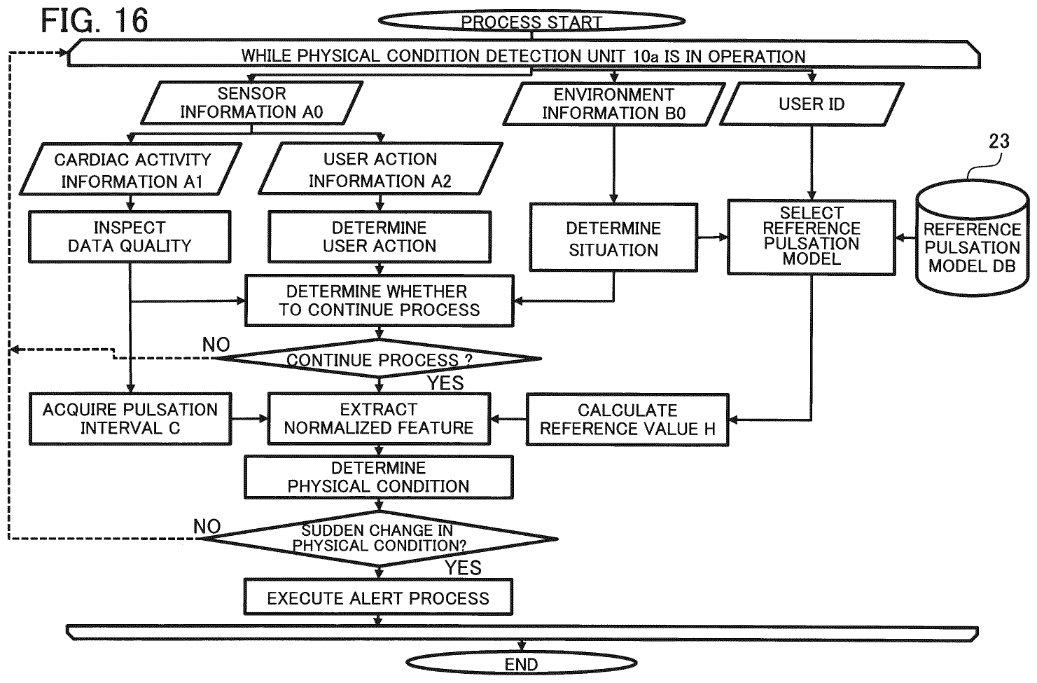

FIG. 15 is a diagram showing a general outline of a process flow executed by the physical condition detection unit 10a in FIG. 12. FIG. 16 is a diagram showing details of the process flow executed by the physical condition detection unit 10a in FIG. 12. The inputs to the physical condition detection device la are the sensor information A0 including the cardiac activity information A1 and the user action information A2, the environment information B0 indicating the situation the user is in, and the user ID for identifying the user using the physical condition detection device 1a. The output from the physical condition detection device 1a is the physical condition determination result F regarding a change in the physical condition.

First, the physical condition detection unit 10a acquires the cardiac activity information A1 and the user action information A2 from the sensor information A0. Subsequently, the process continuation determination unit 11a determines whether the process should be continued or not based on the cardiac activity information A1 and the environment information B0. When it is determined that the process should not be continued, the physical condition detection unit 10a waits for the next input without executing the subsequent processing. When the process is continued, the pulsation interval acquisition unit 12 of the physical condition detection unit 10a acquires the pulsation interval C from the cardiac activity information A1. Subsequently, the reference pulsation calculation unit 24 calculates the reference pulsation for the extraction of the normalized feature. The reference pulsation calculation unit 24 acquires the reference pulsation information corresponding to the situation of the user based on the user ID and the situation the user is in acquired from the situation determination unit 15. Subsequently, the normalized feature extraction unit 13 of the physical condition detection unit 10a extracts the normalized feature by using the acquired pulsation interval C and reference pulsation information. Finally, the physical condition determination result F is outputted by the physical condition determination unit 14 of the physical condition detection unit 10a, and the alert process corresponding to the physical condition detection device 1a is executed when it is determined that there is a sudden change in the physical condition. As examples of the alert process, it is possible to consider a process of displaying a warning, a process of outputting a warning sound, a process of autonomously stopping the vehicle at a safe place in cases of performing autonomous driving, and so forth. The above-described processing is repeated until the process by the physical condition detection device ends.

### (2-5) Effect

As described above, according to the second embodiment, the corrected feature value E is generated by correcting the influence of the individual difference in the pulsation information on the feature value and the influence of a change in the situation the user is in on the feature value, and the determination on a change in the physical condition (e.g., a sudden change in the physical condition, namely, a sudden deterioration in the physical condition) is made based on the corrected feature value E, and thus physical condition detection robust to the individual difference in the pulsation interval C and a change in the situation the user is in can be realized.

### (3) Third Embodiment

In a third embodiment, a description will be given of a physical condition detection device, a physical condition detection system, a physical condition detection method and a physical condition detection program used for detecting a change in the physical condition (e.g., a sudden change in the physical condition, namely, a sudden deterioration in the physical condition) of the user by using a corrected feature value E obtained by correcting the influence of the individual difference in the pulsation interval C on the feature value and an influence of temporal variation in the pulsation interval of the user as the subject on the feature value.

### (3-1) Physical Condition Detection System

The HW configuration of the physical condition detection system according to the third embodiment is similar to that in the first embodiment (shown in FIG. 1). The physical condition detection system according to the third embodiment includes a physical condition detection device 1b and the sensor 103. The physical condition detection system according to the third embodiment may include the input device 104, the storage 105 and the display device 106. The physical condition detection device 1b is a device capable of executing a physical condition detection method according to the third embodiment. Alternatively, the physical condition detection device 1b is a computer capable of executing a physical condition detection program according to the third embodiment.

### (3-2) Physical Condition Detection Device 1b

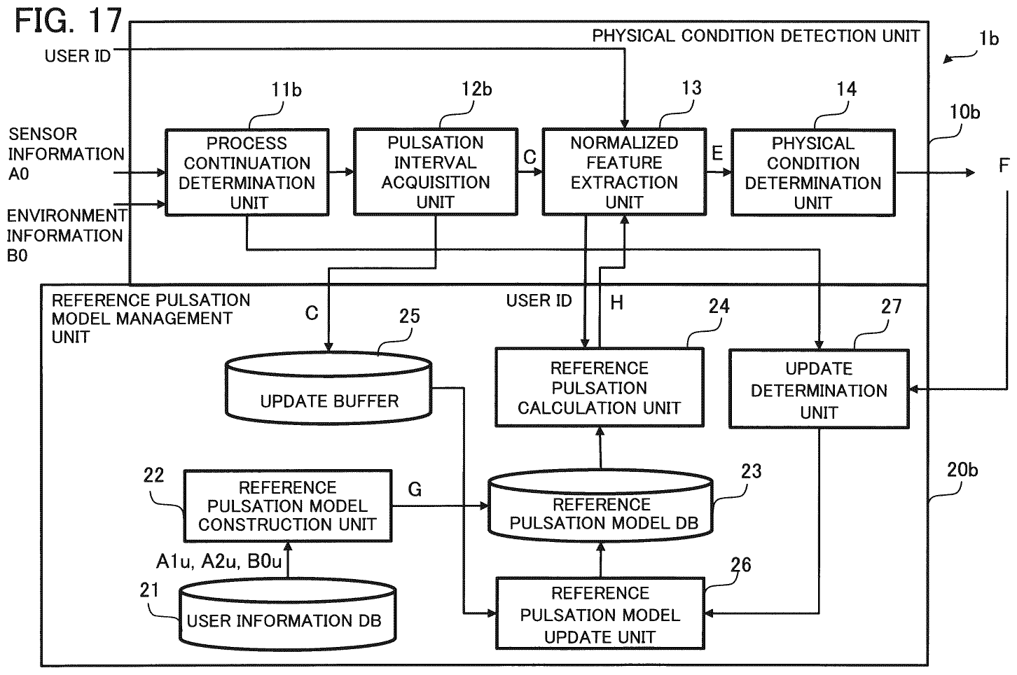

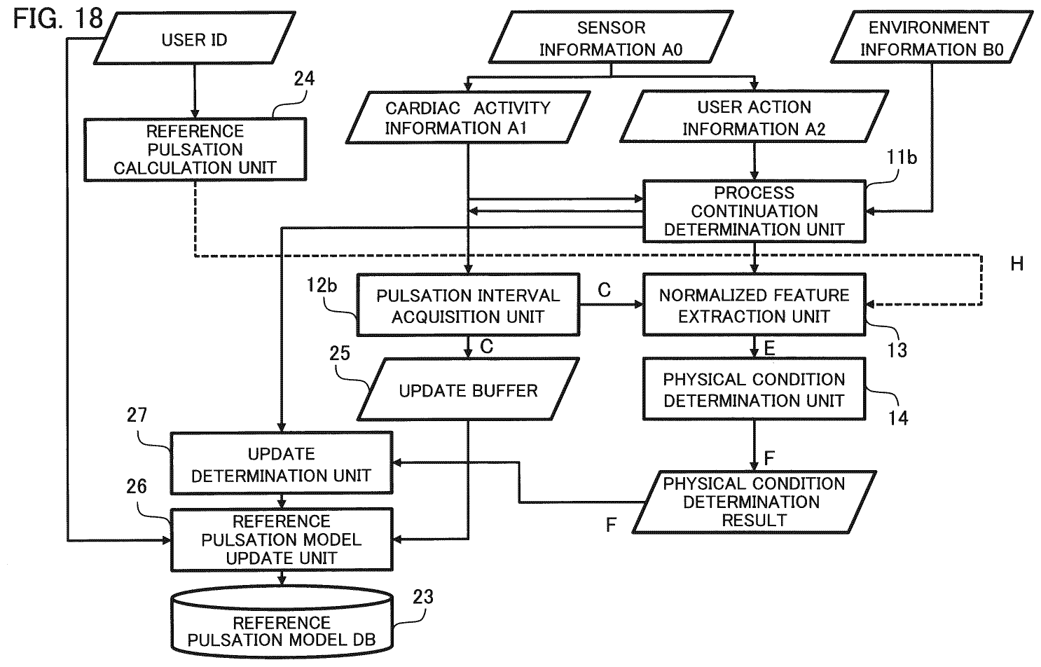

FIG. 17 is a block diagram showing the functional configuration of the physical condition detection device 1b according to the third embodiment. Similarly to the physical condition detection device 1 according to the first embodiment, the physical condition detection device 1b according to the third embodiment includes a reference pulsation model management unit 20b for acquiring the reference information regarding each user as a subject and a physical condition detection unit 10b that detects a sudden change in the physical condition by using the pulsation information acquired from the sensor 103 and the environment information B0 indicating the environment surrounding the user as the subject as inputs. The reference pulsation model management unit 20b includes the reference pulsation model DB 23, the user information DB 21, an update buffer 25, the reference pulsation model construction unit 22, the reference pulsation calculation unit 24, a reference pulsation model update unit 26 and an update determination unit 27. The physical condition detection unit 10b includes a process continuation determination unit 11b, a pulsation interval acquisition unit 12b, the normalized feature extraction unit 13 and the physical condition determination unit 14. The physical condition detection device 1b according to the third embodiment differs from the physical condition detection device 1 according to the first embodiment in that the reference pulsation model G is configured so as to calculate the reference value H having taken the temporal variation in the pulsation interval into account and in that a framework for updating the reference pulsation model G in real time when the physical condition detection unit 10b is used is added. According to the third embodiment, the corrected feature value E in which the correction has been made in regard to not only the influence of the individual difference in the pulsation interval C on the feature value but also the influence of the temporal variation in the pulsation interval C on the feature value can be extracted.

In the following, details of each block will be described. Incidentally, in regard to each of the process continuation determination unit 11b, the pulsation interval acquisition unit 12b, the normalized feature extraction unit 13 and the physical condition determination unit 14 in the physical condition detection unit 10b, the configuration and the process are the same as those in the first embodiment.

### (3-3) Reference Pulsation Model Management Unit 20b

The reference pulsation model management unit 20b in the third embodiment is configured by adding the update buffer 25, the update determination unit 27 and the reference pulsation model update unit 26, as the framework for updating the reference pulsation model G in real time, to the reference pulsation model management unit 20 in the first embodiment. Details regarding each process will be described below.

### Reference Pulsation Model Construction Unit 22

The configuration of the reference pulsation model construction unit 22 in the third embodiment is similar to that of the reference pulsation model construction unit 22 in the first embodiment shown in FIG. 4. The reference pulsation model construction unit 22 in the third embodiment differs from the reference pulsation model construction unit 22 in the first embodiment in using a time-series model as the reference pulsation model G. Since the reference pulsation model G is updated in real time by the model update unit as a subsequent stage, the reference pulsation model G is constructed with a model being updatable and capable of representing the temporal variation. A linear Gaussian state space model can be taken as an example of the reference pulsation model G in the third embodiment. Also in this case, the model is calculated by using information included in the user information DB 21 by the same procedure as that in the first embodiment. Incidentally, the type of the model is not limited as long as the model can be updated sequentially and is capable of representing the temporal variation. Further, in the third embodiment, a configuration excluding the reference pulsation model construction unit 22 is also permissible. It is also permissible to employ a configuration that constructs and updates the reference pulsation model G by exclusively using information acquired when the physical condition detection unit 10b is used, without making the reference pulsation model construction unit 22 previously construct the model.

### Update Buffer 25

The update buffer 25 is an area for temporarily storing a measured time (of day) and the cardiac activity information A1 to be used for updating the reference pulsation model G. The update buffer 25 is an area reserved for at least one input. The amount of data stored in the update buffer 25 varies depending on the frequency of the update of the reference pulsation model G and the amount of data used for the update. Information corresponding to one input is stored in the update buffer 25 at each time in cases where the specification has been set so as to update the reference pulsation model G upon each input, whereas data for 5 minutes is constantly stored in the update buffer 25 in cases where the specification has been set so as to update the reference pulsation model G once in 5 minutes by using data for 5 minutes.

When the update determination unit 27 described below has determined to update the reference pulsation model G, the reference pulsation model G is updated by using the information stored in the update buffer 25.

### Update Determination Unit 27

Whether to update the reference pulsation model G or not is determined by using the cardiac activity information A1 measured by the physical condition detection unit 10b. The measured cardiac activity information A1 has a possibility of deterioration in its data quality due to the user's body motion or the situation the user is in. If the reference pulsation model G is updated by using the whole of the cardiac activity information A1 including such deteriorated information, there is a possibility that a reference value H with high reliability cannot be modeled. The update determination unit 27 determines whether to update the reference pulsation model G at that time or not, which makes it possible to update the reference pulsation model G with appropriate information. Further, the update determination unit 27 determines whether to make the update or not by using not only the inspection of information but also information on the physical condition determination result F obtained from the inspection result. Since the reference pulsation model G is a model representing the reference of each user, namely, the reference pulsation information at the time with no sudden change in the physical condition, the cardiac activity information A1 with which the physical condition detection system determined that there is a sudden change in the physical condition is not used for the update of the reference pulsation model G. Details of the update determination unit 27 will be described below.

The update determination unit 27 determines whether to make the update or not by using the result of the process continuation determination unit 11b in the physical condition detection unit 10b and the physical condition determination result F. Specifically, whether to make the update of the reference pulsation model G or not is determined based on the results of the cardiac activity information inspection unit 111, the user action determination unit 113 and the situation determination unit 112 included in the process continuation determination unit 11b and the physical condition determination result F of the physical condition detection unit 10b. As an example of the update determination, it is determined to update the reference pulsation model G if “the process continuation determination unit 11b determines to continue the process” and “the output of the physical condition detection unit 10b is not a sudden change in the physical condition”. In this case, the reference pulsation model G is consequently updated by using the cardiac activity information A1 with which it is determined that there is no sudden change in the physical condition, included in the cardiac activity information A1 on which the detection process is performed by the physical condition detection unit 10b.

As another example, there is a method in which a different rule for the model update is set by using the results acquired from the cardiac activity information inspection unit 111, the user action determination unit 113 and the situation determination unit 112 included in the process continuation determination unit 11b, without directly using the result of the process continuation determination unit 11b. This method is employed when the rule for updating the reference pulsation model G is desired to be set stricter than the rule regarding the continuation of the detection process, or conversely, looser than the rule regarding the continuation of the detection process.

The update determination unit 27 determines whether to make the update of the reference pulsation model G or not in consideration of the cardiac activity information A1, the user action, and the situation the user is in acquired by the physical condition detection unit 10b in real time and the presence/absence of a sudden change in the physical condition determined by the physical condition detection device.

Incidentally, while the input of the sensor information A0 and the environment information B0 is assumed as a precondition in this process, the process functions even if the process is configured while excluding the environment information B0. In that case, the situation determination unit 112 is excluded from the process continuation determination unit 11b.

Further, the frequency of the update determination may be set arbitrarily. The update determination is made upon each input of data in cases where information at any time acquired by the physical condition detection unit 10b is used for the update, whereas the update determination is made once in 5 minutes in cases where the specification has been set so as to make the update every 5 minutes, for example.

As an example of arbitrarily setting the rule for the update determination, it is possible to consider a method in which the reference pulsation model G is necessarily updated when the situation the user is in is changed. Further, when a physical condition detection device that estimates the condition of the user separately from the physical condition detection unit 10b is in operation at the same time, a detection result of the separate system may be used for the update determination of the reference pulsation model G. A detection result of sleepiness detection, a detection result of wakefulness level detection, an estimation result of emotion estimation, and so forth can be considered as examples of information used for the update.

### Reference Pulsation Model Update Unit 26

When the update determination unit 27 has determined to update the reference pulsation model G, the reference pulsation model G of the relevant user is updated. Data to be used for the update is acquired from the update buffer 25. Since the reference pulsation model G in the third embodiment is formed with a time-series model such as the linear Gaussian state space model, the time-series model is updated by using the pulsation interval acquired from the update buffer 25. The method of updating the reference pulsation model G is not limited to the above-described method. In cases where the linear Gaussian state space model is assumed, the update can be implemented by using a time-series model estimation method such as a Kalman filter or a particle filter.

Further, since the physical condition determination result F of the physical condition detection unit 10b is used by the update determination unit 27 for the update determination, the update of the reference pulsation model G is carried out by using information in the physical condition detection unit 10b one input before at the earliest.

### Reference Pulsation Calculation Unit 24

The reference pulsation calculation unit 24 in the third embodiment receives the user ID as an input and calculates the reference value H of the user “at the present time point”. Since the reference pulsation model G in the third embodiment is formed with a time-series model, the reference pulsation to be inputted to the normalized feature extraction unit 13 is obtained by sampling the pulsation interval information at the present time point predicted by using the reference pulsation model G. Similarly to the first and second embodiments, the specification may be set either to output one value or to output a plurality of candidates.

By making the normalized feature extraction unit 13 perform the normalization of the feature value by using the reference pulsation obtained by the reference pulsation calculation unit 24 in the third embodiment, it is possible to extract the corrected feature value E from which the influence of the individual difference in the pulsation interval on the feature value and the influence of the temporal variation in the pulsation interval on the feature value have been removed.

### (3-4) Physical Condition Detection Unit 10b

The physical condition detection unit 10b in the third embodiment differs from the physical condition detection unit 10 in the first embodiment in that the determination result of the process continuation determination unit 11b is used for the update determination of the reference pulsation model G, the reference value H used by the normalized feature extraction unit 13 is the reference value H having taken the temporal variation into account, and the physical condition determination result F is used for the update determination of the reference pulsation model G. Except for these features, the physical condition detection unit 10b in the third embodiment is the same as the physical condition detection unit 10 in the first embodiment.

### Physical Condition Detection Unit 10b and Reference Pulsation Model G Update Process

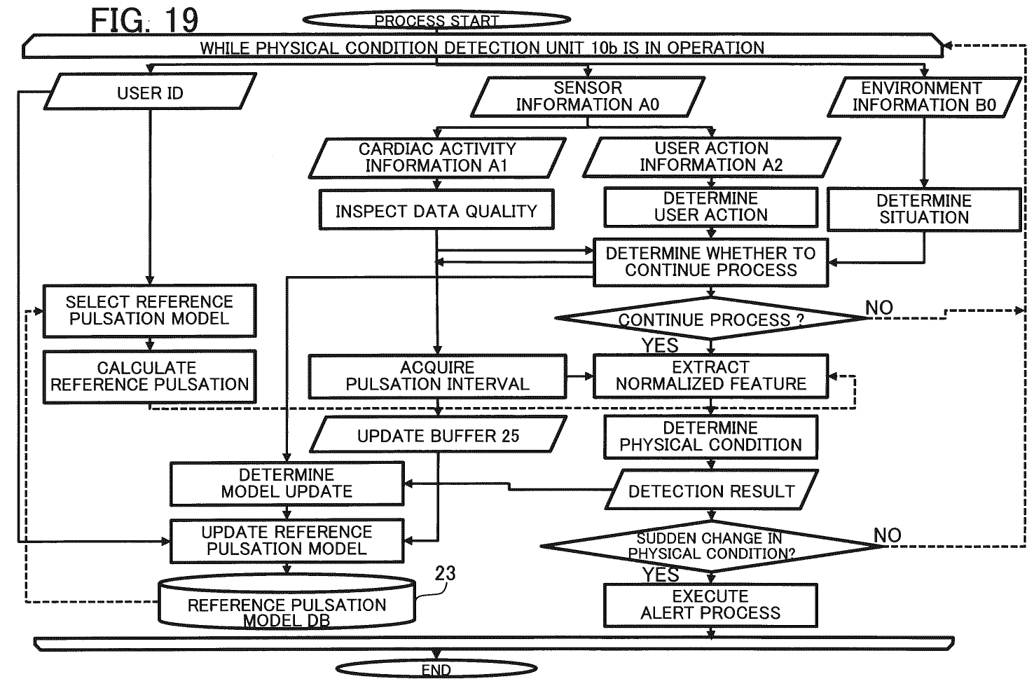

FIG. 18 is a diagram showing a general outline of a process flow executed by the physical condition detection unit 10b in FIG. 17. FIG. 19 is a diagram showing details of the process flow executed by the physical condition detection unit 10b in FIG. 17. The inputs to the physical condition detection device 1b are the sensor information A0 including the cardiac activity information A1 and the user action information A2, the environment information B0 for determining the situation the user is in, and the user ID for identifying the user using the physical condition detection unit 10b. The output from the physical condition detection device 1b is the physical condition determination result F. Further, the physical condition detection device 1b according to the third embodiment does not necessarily require the input of the environment information B0, and may be configured while excluding the input of the environment information B0 and the situation determination unit 15 using the environment information B0 as the input.

First, the physical condition detection unit 10b acquires the cardiac activity information A1 and the user action information A2 from the sensor information A0. Subsequently, the cardiac activity information inspection unit 111 included in the process continuation determination unit 11b inspects the cardiac activity information A1, and when it is determined that the information has no problem (i.e., satisfies a predetermined condition), stores the time-series information on the pulsation interval C calculated by the pulsation interval acquisition unit 12b in the pulsation interval buffer 225. Further, the cardiac activity information inspection unit 111 also stores time information linked with the pulsation interval C in the update buffer 25 at the same time. In parallel with the storing in the update buffer 25, the process continuation determination unit 11b determines whether the physical condition determination process should be continued or not, and when it is determined not to continue the process, waits for the next input without executing the subsequent processing. When the process is continued, the process moves on to the feature extraction process. Further, the result of the process continuation determination unit 11b is used by the update determination unit 27 as a subsequent stage.

In the feature extraction process, first, the reference pulsation is calculated for the extraction of the normalized feature. Here, the reference pulsation calculation unit 24 receives the user ID and a measurement time as inputs and acquires the user's reference pulsation information corresponding to that time point. Subsequently, the normalized feature extraction unit 13 extracts the corrected feature value E as the normalized feature value by using the acquired pulsation interval C and reference pulsation information. The presence/absence of a sudden change in the physical condition is determined by the physical condition determination unit 14 based on the obtained normalized feature value. When it is determined that there is a sudden change in the physical condition, the alert process corresponding to the physical condition detection device is executed. As examples of the alert process, there are a process of displaying a warning on the display device 106 (FIG. 1), a process of outputting a warning sound from a speaker (not shown), a process of making a vehicle autonomously move to a safe place and stop in cases of a vehicle performing autonomous driving, and so forth.

At the time point when the physical condition determination result F is obtained, the process moves on to the reference pulsation model G update process, and the reference pulsation model G update process is executed in parallel with the detection process. The physical condition determination result F is inputted to the update determination unit 27 and whether to make the update of the reference pulsation model G at that time or not is determined based on the physical condition determination result F and the result of the process continuation determination unit 11b put together. When the reference pulsation model G is not updated, the reference pulsation model G update process is ended. When the reference pulsation model G is updated, the reference pulsation model G of the user using the physical condition detection unit 10b is updated by the reference pulsation model update unit 26 by using the pulsation interval C and the measurement time stored in the update buffer 25, and information is stored in the reference pulsation model DB 23. The process described above is repeated until the physical condition detection device ends its process.

### (3-5) Effect

As described above, according to the third embodiment, the corrected feature value E is generated by correcting the influence of the individual difference in the pulsation interval C on the feature value and the influence of the temporal variation in the pulsation interval C on the feature value, and the determination on a change in the physical condition (e.g., a sudden change in the physical condition, namely, a sudden deterioration in the physical condition) is made based on the corrected feature value E, and thus physical condition detection robust to the individual difference and the temporal variation in the pulsation interval C can be realized.

### (4) Fourth Embodiment

In a fourth embodiment, a description will be given of a physical condition detection device 1c, a physical condition detection system, a physical condition detection method and a physical condition detection program for detecting a change in the physical condition (e.g., a sudden change in the physical condition, namely, a sudden deterioration in the physical condition) of the user by using a corrected feature value E obtained by correcting the influence of the individual difference in the pulsation interval on the feature value, the influence of a change in the situation the user as the subject is in on the feature value, and the influence of the temporal variation in the pulsation interval on the feature value.

### (4-1) Physical Condition Detection System

The HW configuration of the physical condition detection system according to the fourth embodiment is similar to that in the first embodiment (FIG. 1). The physical condition detection system according to the fourth embodiment includes the physical condition detection device 1c and the sensor 103. The physical condition detection system according to the fourth embodiment may include the input device 104, the storage 105 and the display device 106. The physical condition detection device 1c is a device capable of executing a physical condition detection method according to the fourth embodiment. Alternatively, the physical condition detection device 1c is a computer capable of executing a physical condition detection program according to the fourth embodiment.

### (4-2) Physical Condition Detection Device 1c

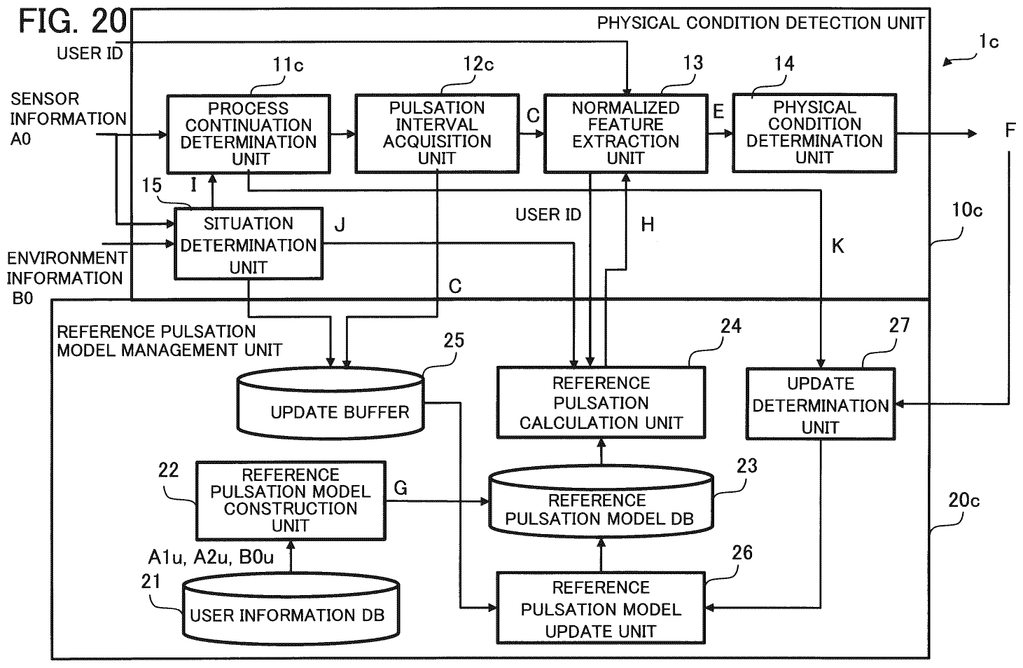

FIG. 20 is a block diagram showing the functional configuration of the physical condition detection device 1c according to the fourth embodiment. Similarly to the physical condition detection device 1 in the first embodiment, the physical condition detection device 1c according to the fourth embodiment includes a reference pulsation model management unit 20c for acquiring the reference information regarding each user as a subject and calculating the reference pulsation model G of each user and a physical condition detection unit 10c that detects a change in the physical condition (e.g., a sudden change in the physical condition, namely, a sudden deterioration in the physical condition) by using the pulsation information as the sensor information A0 acquired from the sensor 103 (FIG. 1) and the environment information B0 indicating the environment surrounding the user as the subject as inputs. The reference pulsation model management unit 20c includes the reference pulsation model DB 23, the user information DB 21, the update buffer 25, the reference pulsation model construction unit 22, the reference pulsation calculation unit 24, the reference pulsation model update unit 26 and the update determination unit 27. The physical condition detection unit 10c includes a process continuation determination unit 11c, the situation determination unit 15, a pulsation interval acquisition unit 12c, the normalized feature extraction unit 13 and the physical condition determination unit 14.

The physical condition detection device 1c according to the fourth embodiment detects a change in the physical condition by using the corrected feature value E obtained by correcting the influence of the individual difference in the pulsation interval C on the feature value (described in the first embodiment), correcting the influence of a change in the situation the user as the subject is in on the feature value (described in the second embodiment), and correcting the influence of the temporal variation in the pulsation interval C on the feature value (described in the third embodiment). In other words, the physical condition detection device 1c according to the fourth embodiment makes the determination on a change in the physical condition by using the corrected feature value E obtained by making the correction of the influence of the situation the user is in in the second embodiment and also making the correction of the influence of the temporal variation in the pulsation interval C on the feature value in the third embodiment.

Incidentally, each of the process continuation determination unit 11c, the pulsation interval acquisition unit 12c, the normalized feature extraction unit 13 and the physical condition determination unit 14 in the physical condition detection unit 10c has a configuration similar to that of a corresponding unit in the physical condition detection unit 10 in the first embodiment. The physical condition detection device 1c according to the fourth embodiment differs from the physical condition detection device 1b according to the third embodiment in that the situation determination result J obtained by the situation determination unit 15 is used for the calculation of the reference pulsation and the reference pulsation model G is made to correspond to not only the temporal variation but also a change in the situation.

### (4-3) Reference Pulsation Model Management Unit 20c

The reference pulsation model management unit 20c in the fourth embodiment is a unit obtained by adding a framework for constructing and updating the reference pulsation model G having taken the influence of a change in the situation the user is in on the feature value into account to the reference pulsation model management unit 20b in the third embodiment.

### Reference Pulsation Model Construction Unit 22

The configuration of the reference pulsation model construction unit 22 in the fourth embodiment is similar to that in the third embodiment. The reference pulsation model construction unit 22 in the fourth embodiment differs from the reference pulsation model construction unit 22 in the third embodiment in that a time-series model having taken the influence of a change in the situation the user is in on the feature value into account is used as the reference pulsation model G. Further, accordingly, information regarding the situation determined by the situation determination unit 15 is stored in the update buffer 25 in addition to the cardiac activity information A1 and the measurement time. Here, a description will be given of an example in which there exist four situations: situations #1 to #4, as the situations assumed by the physical condition detection device and “the situation the user is in” when the reference pulsation model G is handled is already known. Expression (6) indicates an example of the reference pulsation model (Xₙ) in the situation #n. Expressions (7), (8) and (9) respectively indicate an observation noise model d representing noise in the observed pulsation intervals, a temporal variation model tₙ representing the temporal variation in the pulsation interval, and a situation influence model sₙ representing the influence of a change in the situation.

$${X}_{n} = {t}_{n} + {s}_{n} + {d}_{n} \qquad ( 6 )$$

$${d}_{n} ∼ N ( 0 , {σ}_{y}^{2} ) \qquad ( 7 )$$

$${t}_{n} = {\sum }_{i = 1}^{k} {c}_{i}^{( k )} {t}_{n - 1} + {e}_{n} \qquad ( 8 )$$

$${s}_{n} = {\sum }_{i = 1}^{N} {β}_{n}^{( i )} {x}_{n}^{( i )} + {f}_{n} \qquad ( 9 )$$

The character k included in the temporal variation model tₙ represents the degree of a trend component indicating the temporal variation, and is a value that means conditions up to how many times ago should be referred to in order to estimate the trend. The character cᵢ^{(k)} is a coefficient that varies depending on the degree of the trend component indicating the temporal variation. The character βₙ⁽ⁱ⁾ included in the situation influence model sₙ is a coefficient indicating the influence of the situation #i on the pulsation interval, and xₙ⁽ⁱ⁾ represents the situation the user is in calculated by the situation determination unit 15. For example, when the situation in the input data is the “situation #1”, xₙ⁽ⁱ⁾ takes on the following values:

$${x}_{n}^{( 1 )} = 1 , {x}_{n}^{( 2 )} = 0 , {x}_{n}^{( 3 )} = 0 , {x}_{n}^{( 4 )} = 0$$

Further, when the situation in the input data is the “situation #2”, i=2 and xₙ⁽ⁱ⁾ takes on the following values:

$${x}_{n}^{( 1 )} = 0 , {x}_{n}^{( 2 )} = 1 , {x}_{n}^{( 3 )} = 0 , {x}_{n}^{( 4 )} = 0$$

Similarly, only xₙ⁽³⁾ equals 1 when i=3, and only xₙ⁽⁴⁾ equals 1 when i=4.

The term eₙ is a noise component occurring in the temporal variation model tₙ and is represented by expression (10). The term fₙ is a noise component in the situation influence model sₙ and is represented by expression (11).

$${e}_{n} ∼ N ( 0 , {σ}_{t}^{2} ) \qquad ( 10 )$$

$${f}_{n} ∼ N ( 0 , {σ}_{s}^{2} ) \qquad ( 11 )$$

The above-described examples are examples on the assumption that each noise component is in Gaussian distribution. Also in the construction of the reference pulsation model G in the fourth embodiment, the reference pulsation model G is calculated by estimating various parameters of the reference pulsation model G by using information included in the user information DB 21 according to a procedure similar to that in the first embodiment. Incidentally, the type and the construction method of the reference pulsation model G are not limited to the above-described examples as long as the reference pulsation model G is a reference pulsation model G that can be updated sequentially and is capable of representing the influence of the temporal variation and the situation. Further, in the fourth embodiment, a configuration excluding the reference pulsation model construction unit 22 is also permissible similarly to the third embodiment. It is also permissible to employ a configuration that constructs and updates the reference pulsation model G by exclusively using information acquired when the physical condition detection unit 10c is used, without making the reference pulsation model construction unit 22 previously construct the reference pulsation model G.

### Update Buffer 25

The update buffer 25 in the fourth embodiment differs from the update buffer in the third embodiment in storing the information regarding the situation calculated by the situation determination unit 15 in addition to the cardiac activity information A1 and the measurement time.

### Update Determination Unit 27

The update determination unit 27 in the fourth embodiment has a configuration similar to that in the third embodiment and executes a similar process.

### Reference Pulsation Model Update Unit 26

The reference pulsation model update unit 26 in the fourth embodiment updates the reference pulsation model G represented by a time-series model by using the information stored in the update buffer 25 similarly to the reference pulsation model update unit 26 in the third embodiment. However, the reference pulsation model update unit 26 in the fourth embodiment differs from that in the third embodiment and updates the reference pulsation model G by using the situation determination result J in addition to the cardiac activity information A1 and the measurement time.

### Reference Pulsation Calculation Unit 24

The reference pulsation calculation unit 24 in the fourth embodiment receives the user ID as an input and calculates the reference value H of the user identified by the user ID “at the present time point”. The reference pulsation model G in the fourth embodiment is formed with a time-series model similarly to the reference pulsation model G in the third embodiment. Therefore, the reference pulsation calculation unit 24 in the fourth embodiment can use the pulsation interval C at the present time point predicted by using the reference pulsation model G as the reference pulsation as the reference value H to be inputted to the normalized feature extraction unit 13. The reference pulsation calculation unit 24 in the fourth embodiment may be configured either to output one value or to output a plurality of candidate values similarly to the reference pulsation calculation units 24 in the first to third embodiments.

The normalized feature extraction unit 13 performs the normalization of the feature value by using the reference pulsation obtained by the reference pulsation calculation unit 24 in the fourth embodiment, and thus is capable of extracting the corrected feature value E from which the influence of the individual difference in the pulsation interval C on the feature value, the influence of the temporal variation in the pulsation interval C on the feature value, and the influence of the situation the user is in on the feature value have been removed.

### (4-4) Physical Condition Detection Unit 10c

The configuration of the physical condition detection unit 10c in the fourth embodiment is similar to that of the physical condition detection unit 10a in the second embodiment. The physical condition detection unit 10c in the fourth embodiment differs from the physical condition detection unit 10a in the second embodiment in that the process continuation determination result K of the process continuation determination unit 11c is used for the update determination of the reference pulsation model G, the reference value H used by the normalized feature extraction unit 13 is the reference value H having taken the temporal variation into account, and the physical condition determination result F is used for the update determination of the reference pulsation model G.

### Physical Condition Detection Unit 10c and Reference Pulsation Model G Update Process

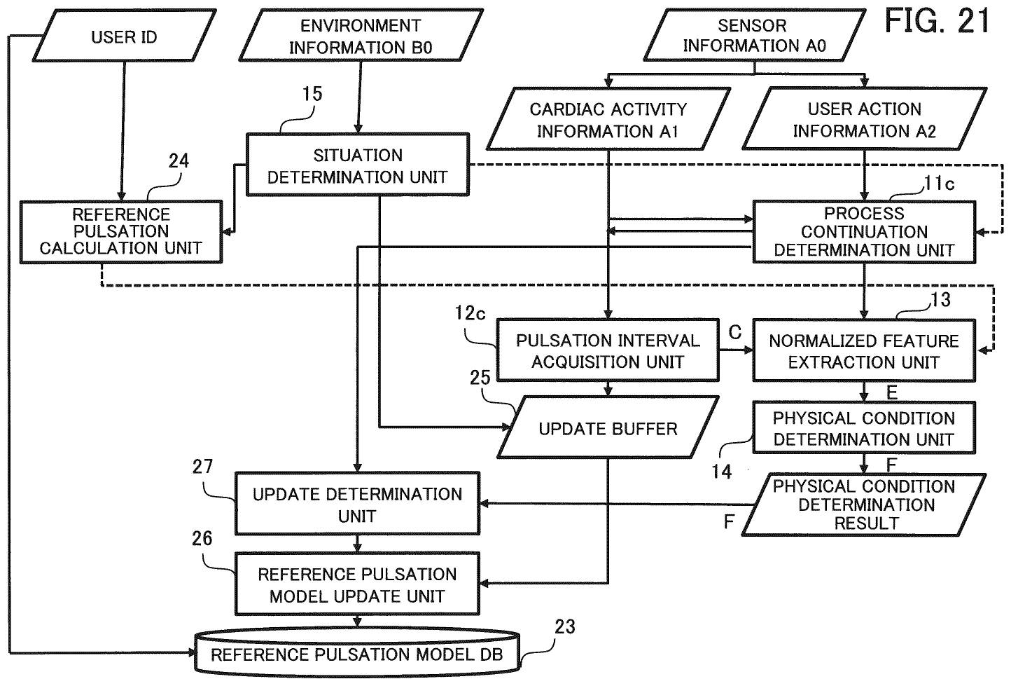

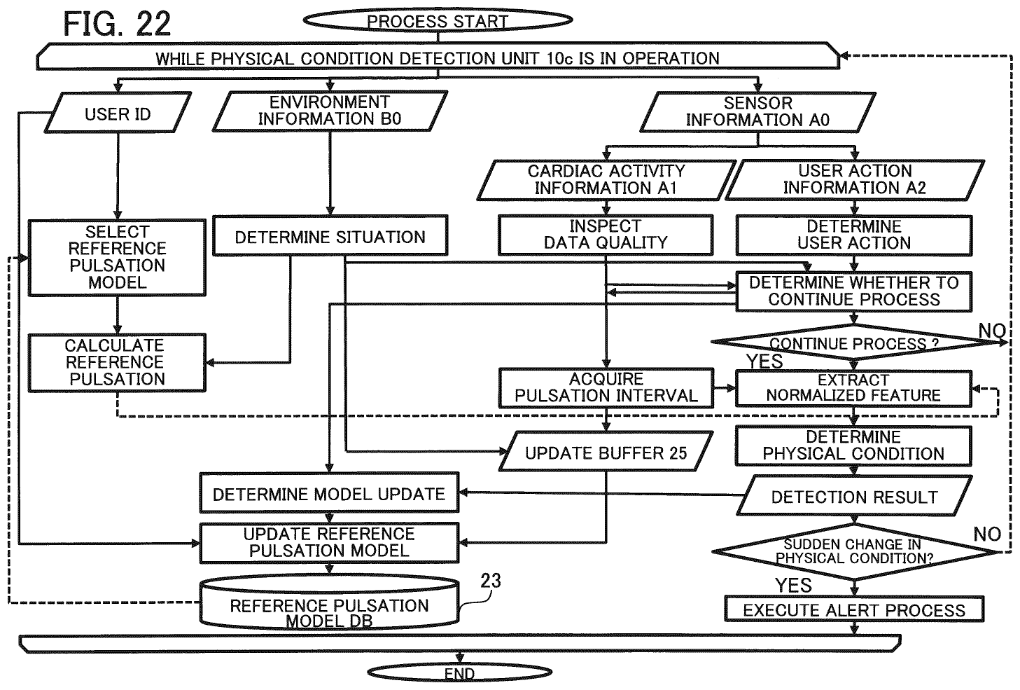

FIG. 21 is a diagram showing a general outline of a process flow executed by the physical condition detection unit 10c in FIG. 20. FIG. 22 is a diagram showing details of the process flow executed by the physical condition detection unit 10c in FIG. 20. The inputs to the physical condition detection unit 10c are the sensor information A0 including the cardiac activity information A1 and the user action information A2, the environment information B0 indicating the situation the user is in, and the user ID for identifying the user using the physical condition detection unit 10c. The output from the physical condition detection unit 10c is the physical condition determination result F as the determination result regarding a sudden change in the physical condition of the user.

First, the physical condition detection unit 10c acquires the cardiac activity information A1 and the user action information A2 from the sensor information A0. Subsequently, the cardiac activity information inspection unit 111 included in the process continuation determination unit 11c inspects the cardiac activity information A1, and when it is determined that the cardiac activity information A1 has no problem (i.e., satisfies a predetermined condition), stores the time-series information on the pulsation interval C calculated by the pulsation interval acquisition unit 12c in the pulsation interval buffer 225. In this case, the cardiac activity information inspection unit 111 also stores time information linked with the pulsation interval C in the update buffer 25 at the same time.

Further, the process continuation determination unit 11c also stores “the determination result regarding the situation the user is in” obtained by the situation determination unit 15 in the update buffer 25. In parallel with the storing in the update buffer 25, the process continuation determination unit 11c determines whether the physical condition detection process should be continued or not, and when it is determined not to continue the process, waits for the next input without executing the subsequent processing. When the process continuation determination unit 11c determines to continue the physical condition detection process, the process moves on to the feature extraction process. Further, the determination result of the process continuation determination unit 11c is used by the update determination unit 27 as a subsequent stage.

In the feature extraction process, the normalized feature extraction unit 13 calculates the reference pulsation for the extraction of the normalized feature. Here, the reference pulsation calculation unit 24 acquires the user's reference pulsation corresponding to that time point based on the user ID, the measurement time and the situation determination result. Subsequently, the normalized feature extraction unit 13 extracts the normalized feature value by using the acquired pulsation interval C and reference pulsation. The physical condition determination unit 14 determines the presence/absence of a sudden change in the physical condition based on the corrected feature value E as the obtained normalized feature value. When the physical condition determination unit 14 determines that there is a sudden change in the physical condition, the alert process corresponding to the physical condition detection device 1c is executed. As examples of the alert process, there are a process of displaying a warning on the display device 106, a process of outputting a warning sound from a speaker (not shown), a process of making a vehicle autonomously move to a safe place and stop in cases of a vehicle performing autonomous vehicle driving, and so forth.

At the time point when the physical condition determination result F is obtained, the reference pulsation model management unit 20c moves on to the reference pulsation model G update process, and the reference pulsation model G update process is executed in parallel with the detection process. When the physical condition determination result F is inputted to the update determination unit 27, the update determination unit 27 determines whether to make the update of the reference pulsation model G at that time or not based on the physical condition determination result F and the process continuation determination result K of the process continuation determination unit 11c put together. When the reference pulsation model G is not updated, the reference pulsation model management unit 20c ends the reference pulsation model G update process. When the reference pulsation model G is updated, the reference pulsation model management unit 20c makes the reference pulsation model update unit 26 update the reference pulsation model G of the user using the physical condition detection unit 10c by using the pulsation interval C, the situation determination result and the measurement time stored in the update buffer 25, and stores the updated reference pulsation model G in the reference pulsation model DB 23. The process described above is repeated until the process by the physical condition detection device 1c ends.

### (4-5) Effect

As described above, according to the fourth embodiment, the corrected feature value E is generated by correcting the influence of the individual difference in the pulsation information on the feature value, the influence of a change in the situation the user is in on the feature value, and the influence of the temporal variation in the pulsation interval C on the feature value, and the determination on a change in the physical condition (e.g., a sudden change in the physical condition, namely, a sudden deterioration in the physical condition) is made based on the corrected feature value E, and thus physical condition detection robust to the individual difference in the pulsation interval C, the temporal variation in the pulsation interval C, and the variation in the pulsation interval C due to a change in the situation can be realized.

### (5) Appendixes

Various aspects of the present disclosure are collectively described below as appendixes.

### Appendix 1

A physical condition detection device that determines a change in physical condition of a subject as an inspection target user identified by user identification information, comprising:
a reference pulsation model construction unit to construct a reference of a pulsation interval of each of one or more users as a reference pulsation model in regard to each user;
a reference pulsation calculation unit to select the reference pulsation model of the subject from the reference pulsation models and to generate a reference value indicating the pulsation interval of the subject from the selected reference pulsation model;
a normalized feature extraction unit to receive time-series pulsation information being information regarding the subject detected by a sensor, to extract a feature value of a real-time pulsation interval from the time-series pulsation information, and to correct the feature value by normalizing the feature value by using the reference value, thereby generating a real-time corrected feature value; and
a physical condition determination unit to determine a change in the physical condition of the subject based on the real-time corrected feature value.

### Appendix 2

The physical condition detection device according to appendix 1, wherein
the reference pulsation model construction unit constructs the reference of the pulsation interval of each user and a situation each user is in as the reference pulsation model in regard to each user, and
the reference pulsation calculation unit selects the reference pulsation model of the subject from the reference pulsation models and generates the reference value indicating the pulsation interval of the subject and the situation the subject is in from the selected reference pulsation model.

### Appendix 3

The physical condition detection device according to appendix 1, wherein
the reference pulsation model construction unit constructs the reference of the pulsation interval of each user and user action information indicating an action of each user as the reference pulsation model in regard to each user, and
the reference pulsation calculation unit selects the reference pulsation model of the subject from the reference pulsation models and generates the reference value indicating the pulsation interval of the subject and the action of the subject from the selected reference pulsation model.

### Appendix 4

The physical condition detection device according to appendix 1, wherein
the reference pulsation model construction unit constructs the reference of the pulsation interval of each user, a situation each user is in, and user action information indicating an action of each user as the reference pulsation model in regard to each user, and
the reference pulsation calculation unit selects the reference pulsation model of the subject from the reference pulsation models and generates the reference value indicating the pulsation interval of the subject, the situation the subject is in, and environment information around each user from the selected reference pulsation model.

### Appendix 5

The physical condition detection device according to any one of appendixes 1 to 4, further comprising:
an update determination unit to determine whether update of the reference pulsation model should be made or not based on a physical condition determination result obtained by the physical condition determination unit; and
a reference pulsation model update unit to update the reference pulsation model in real time when it is determined that the update should be made.

### Appendix 6

The physical condition detection device according to any one of appendixes 1 to 5, wherein the sensor includes a pulsation sensor to detect pulsation of the subject and to output the pulsation information.

### Appendix 7

The physical condition detection device according to appendix 3 or 4, wherein the sensor includes an action sensor to measure the action of the subject and to output the user action information.

### Appendix 8

The physical condition detection device according to appendix 2 or 4, wherein the sensor includes an environment sensor to measure the situation the subject is in and to output environment information.

### Appendix 9

A physical condition detection system comprising:
the physical condition detection device according to any one of appendixes 1 to 8; and the sensor.

### Appendix 10

A physical condition detection method to be executed by a physical condition detection device that determines a change in physical condition of a subject as an inspection target user identified by user identification information, the method comprising:
a step of constructing a reference of a pulsation interval of each of one or more users as a reference pulsation model in regard to each user;
a step of selecting the reference pulsation model of the subject from the reference pulsation models and generating a reference value indicating the pulsation interval of the subject from the selected reference pulsation model;
a step of receiving time-series pulsation information being information regarding the subject detected by a sensor, extracting a feature value of a real-time pulsation interval from the time-series pulsation information, and correcting the feature value by normalizing the feature value by using the reference value, thereby generating a real-time corrected feature value; and
a step of determining a change in the physical condition of the subject based on the real-time corrected feature value.

### Appendix 11

A physical condition detection program that causes a computer, determining a change in physical condition of a subject as an inspection target user identified by user identification information, to execute:
a step of constructing a reference of a pulsation interval of each of one or more users as a reference pulsation model in regard to each user;
a step of selecting the reference pulsation model of the subject from the reference pulsation models and generating a reference value indicating the pulsation interval of the subject from the selected reference pulsation model;
a step of receiving time-series pulsation information being information regarding the subject detected by a sensor, extracting a feature value of a real-time pulsation interval from the time-series pulsation information, and correcting the feature value by normalizing the feature value by using the reference value, thereby generating a real-time corrected feature value; and
a step of determining a change in the physical condition of the subject based on the real-time corrected feature value.

### DESCRIPTION OF REFERENCE CHARACTERS

1, 1a, 1b, 1c: physical condition detection device, 10, 10a, 10b, 10c: physical condition detection unit, 11, 11a, 11b, 11c: process continuation determination unit, 12, 12b, 12c: pulsation interval acquisition unit, 13: normalized feature extraction unit, 14: physical condition determination unit, 15: situation determination unit, 20, 20a, 20b, 20c: reference pulsation model management unit, 21: user information DB, 22: reference pulsation model construction unit, 23: reference pulsation model DB, 24: reference pulsation calculation unit, 25: update buffer, 26: reference pulsation model update unit, 27: update determination unit, 101: processor, 102: memory, 103: sensor, 111: cardiac activity information inspection unit, 112: situation determination unit, 113: user action determination unit, 114: continuation determination unit, 131: feature extraction unit, 132: normalization unit, 220: data inspection unit, 221: cardiac activity information inspection unit, 222: situation determination unit, 223: user action determination unit, 224: data usage determination unit, 225: pulsation interval buffer, 226: model calculation unit, A0: sensor information, A1: cardiac activity information (pulsation information), A2: user action information, B0: environment information, A1u: cardiac activity information (pulsation information), A2u: user action information, B0u: environment information, C: pulsation interval, E: corrected feature value, F: physical condition determination result, G: reference pulsation model, H: reference value, J: situation determination result.

What is claimed is:

1. A physical condition detection device that determines a change in physical condition of a subject as an inspection target user identified by user identification information, comprising:
processing circuitry
to construct any of a reference of a pulsation interval, a reference of a feature value obtained from the pulsation interval, and both of the references of the pulsation interval and the feature value, as reference pulsation models in regard to each user;
to select a reference pulsation model of the subject from the reference pulsation models and to generate any of a reference value indicating the pulsation interval of the subject, a reference value of the feature value obtained from the pulsation interval of the subject, and both of the reference values of the pulsation interval of the subject and the feature value of the subject, as a subject reference value, from the selected reference pulsation model;
to receive time-series pulsation information that is information regarding the subject and is detected by a sensor, to extract a real-time feature value that is a feature value of a real-time pulsation interval from the time-series pulsation information, and to correct the real-time feature value by normalizing the real-time feature value by using the subject reference value, thereby generating a real-time corrected feature value;
to determine a change in the physical condition of the subject based on the real-time corrected feature value;
to determine whether update of the reference pulsation models should be made or not based on a physical condition determination result that is a result of the determining the update of the reference pulsation models; and
to update the reference pulsation models in real time when it is determined that the update should be made.

2. The physical condition detection device according to claim 1, wherein the processing circuitry
constructs the reference of the pulsation interval of each user and a situation each user is in as the reference pulsation model in regard to each user, and
selects the reference pulsation model of the subject from the reference pulsation models and generates the reference value indicating the pulsation interval of the subject and the situation the subject is in from the selected reference pulsation model.

3. The physical condition detection device according to claim 1, wherein the processing circuitry
constructs the reference of the pulsation interval of each user and user action information indicating an action of each user as the reference pulsation model in regard to each user, and
selects the reference pulsation model of the subject from the reference pulsation models and generates the reference value indicating the pulsation interval of the subject and the action of the subject from the selected reference pulsation model.

4. The physical condition detection device according to claim 1, wherein the processing circuitry
constructs the reference of the pulsation interval of each user, a situation each user is in, and user action information indicating an action of each user as the reference pulsation model in regard to each user, and
selects the reference pulsation model of the subject from the reference pulsation models and generates the reference value indicating the pulsation interval of the subject, the situation the subject is in, and environment information around each user from the selected reference pulsation model.

5. The physical condition detection device according to claim 1, wherein the sensor includes a pulsation sensor to detect pulsation of the subject and to output the time-series pulsation information.

6. The physical condition detection device according to claim 3, wherein the sensor includes an action sensor to measure the action of the subject and to output the user action information.

7. The physical condition detection device according to claim 4, wherein the sensor includes an environment sensor to measure the situation the subject is in and to output environment information.

8. A physical condition detection system comprising:
the physical condition detection device according to claim 1; and
the sensor.

9. A physical condition detection system comprising:
the physical condition detection device according to claim 2; and
the sensor.

10. A physical condition detection system comprising:
the physical condition detection device according to claim 3; and
the sensor.

11. A physical condition detection system comprising:
the physical condition detection device according to claim 4; and
the sensor.

12. A physical condition detection method to be executed by a physical condition detection device that determines a change in physical condition of a subject as an inspection target user identified by user identification information, the method comprising:
constructing any of a reference of a pulsation interval, a reference of a feature value obtained from the pulsation interval, and both of the references of the pulsation interval and the feature value, as reference pulsation models in regard to each user;
selecting a reference pulsation model of the subject from the reference pulsation models and generating any of a reference value indicating the pulsation interval of the subject, a reference value of the feature value obtained from the pulsation interval of the subject, and both of the reference values of the pulsation interval of the subject and the feature value of the subject, as a subject reference value, from the selected reference pulsation model;
receiving time-series pulsation information that is information regarding the subject and is detected by a sensor, extracting a real-time feature value that is a feature value of a real-time pulsation interval from the time-series pulsation information, and correcting the real-time feature value by normalizing the real-time feature value by using the subject reference value, thereby generating a real-time corrected feature value;
determining a change in the physical condition of the subject based on the real-time corrected feature value;
determining whether update of the reference pulsation model should be made or not based on a physical condition determination result that is a result of the determining of the update of the reference pulsation model; and
updating the reference pulsation model in real time when it is determined that the update should be made.

13. A physical condition detection program that causes a computer, determining a change in physical condition of a subject as an inspection target user identified by user identification information, to execute:
constructing any of a reference of a pulsation interval, a reference of a feature value obtained from the pulsation interval, and both of the references of the pulsation interval and the feature value, as reference pulsation models in regard to each user;
selecting a reference pulsation model of the subject from the reference pulsation models and generating any of a reference value indicating the pulsation interval of the subject, a reference value of the feature value obtained from the pulsation interval of the subject, and both of the reference values of the pulsation interval of the subject and the feature value of the subject, as a subject reference value, from the selected reference pulsation model;
receiving time-series pulsation information that is information regarding the subject and is detected by a sensor, extracting a real-time feature value that is a feature value of a real-time pulsation interval from the time-series pulsation information, and correcting the real-time feature value by normalizing the real-time feature value by using the subject reference value, thereby generating a real-time corrected feature value;
determining a change in the physical condition of the subject based on the real-time corrected feature value;
determining whether update of the reference pulsation model should be made or not based on a physical condition determination result that is a result of the determining of the reference pulsation model; and
updating the reference pulsation model in real time when it is determined that the update should be made.
